(19) United States
(12) Patent Application Publication
(10) Pub. No.: US 2018/0371256 A1
(43) Pub. Date: Dec. 27, 2018

(54) BENZOCOUMARIN-BASED TWO-PHOTON ABSORBING FLUORESCENT DYE

(71) Applicant: POSTECH ACADEMY-INDUSTRY FOUNDATION, Pohang-si (KR)

(72) Inventors: Kyo Han Ahn, Pohang-si (KR); Yong Woong Jun, Pohang-si (KR); Hyerim Kim, Daejeon (KR); Subhankar Singha, Pohang-si (KR); Ye Jin Reo, Hwaseong-si (KR)

(21) Appl. No.: 15/955,877

(22) Filed: Apr. 18, 2018

(30) Foreign Application Priority Data
Jun. 22, 2017 (KR) 10-2017-0078999

(51) Int. Cl. C09B 57/02; G01N 33/58; C09K 11/06

(52) U.S. Cl. CPC C09B 57/02; G01N 33/582; C09K 2211/1007; C09K 2211/1018; C09K 11/06

(57) ABSTRACT

Provided is a method for preparing a novel benzocoumarin-based two-photon absorbing fluorescent dye, such as a pyridinium-benzocoumarin (Py⁺BC) derivative compound and its precursor compound (pyridyl-benzocoumarin, PyBC), an aryl-benzocoumarin (ArBC) derivative compound, a benzothiazolyl-benzocoumarin (BtBC) derivative compound or a keto-benzocoumarin (KetoBC) derivative compound. The Py⁺BC derivative compound of the present invention is a two-photon absorbing near-infrared fluorescent dye, which may minimize interference of autofluorescence compared to conventional two-photon absorbing fluorescent dyes such as acedan and naphthalimide, thereby obtaining a high-definition image, and therefore is expected to be effectively used in imaging studies. In addition, when the two-photon absorbing near-infrared fluorescent dye of the present invention is used, it is expected to be suitable for high resolution imaging of deep tissue.

### CROSS-REFERENCE TO RELATED APPLICATION

This application claims priority to and the benefit of Korean Patent Application No. 2017-0078999, filed on Jun. 22, 2017, the disclosure of which is incorporated herein by reference in its entirety.

### STATEMENT REGARDING GOVERNMENT RIGHTS

The present invention was undertaken with the support of “Development of Two-photon Fluorescence Probes for Disease Diagnosis and Imaging” No. 2016915035 grant funded by the National Research Foundation of Korea.

### BACKGROUND

### 1. Field of the Invention

The present invention relates to compounds of a novel benzocoumarin-based two-photon absorbing fluorescent dye, which have various substituents at the 3-position, and more particularly, to a method for preparing a pyridinium-benzocoumarin (Py⁺BC) derivative compound and its precursor compound (pyridyl-benzocoumarin, PyBC), an aryl-benzocoumarin (ArBC) derivative compound, a benzothiazolyl-benzocoumarin (BtBC) derivative compound or a keto-benzocoumarin (KetoBC) derivative compound and applications thereof.

### 2. Discussion of Related Art

Bio-imaging techniques based on a fluorescence signal have been widely used as methods for visualizing cell organelles including tissue and the like in an animal model, and among these, development of fluorescent probes widens the scope of their application in analysis and imaging of a specific substance in an organism.

Most fluorescent probes that have been reported to date are manufactured based on one-photon absorbing fluorescent dyes, and used in imaging by one-photon microscopy (OPM). However, in tissue imaging, different from cell imaging, fluorescent probes are not suitable for imaging of deep tissue (tens of micrometers or greater) due to the decrease in image quality by light scattering [Weissleder, R. et al. Curr. Opin. Chem. Biol. 2010, 14, 71; Nie, S. et al. Nat. Nanotechnol. 2009, 4, 710].

However, non-linear optical microscopy is not sensitive to the influence of such light scattering and facilitates high definition imaging on deeper tissue, and therefore is advantageous for tissue imaging. For example, two-photon microscopy (TPM) is one type of non-linear optical microscopy, and characterized by excitation of a fluorophore by applying two photons with energy corresponding to a half of photon energy used in one-photon microscopy (OPM) to the fluorophore at the same time. Such two-photon imaging has advantages of high tissue penetration, low photo-damage to bio tissue because only a focal point is excited, and low photobleaching with respect to a fluorophore in bio-imaging. The two-photon imaging also has advantages of minimized interference by an auto-fluorescent substance in an organism and high-resolution imaging.

For bio-imaging using two-photon microscopy, a two-photon absorbing fluorescent dye with a small molecular weight is required, but only a few substances have been known. Representative examples of such substances are acedan, naphthalimide, and 7-aminocoumarin derivatives. In addition, these fluorescent dyes emit fluorescence in a blue-green range, which overlaps with a fluorescent range caused by a material in an organism, resulting in decreased reliability in tissue imaging. Accordingly, since the fluorescence has a relatively higher intensity than an auto-fluorescent signal in a red wavelength range beyond the autofluorescence range due to a substance in an organism, the reliability of the fluorescent signal in tissue imaging is decreased. Therefore, it is necessary to develop a novel fluorophore capable of overcoming disadvantages of a conventional two-photon absorbing material.

### SUMMARY OF THE INVENTION

To overcome the problems of the conventional art, the inventors developed a novel two-photon absorbing fluorescent dye capable of realizing high-resolution tissue imaging by minimizing the interference of autofluorescence, and thus the present invention was completed. The two-photon absorbing fluorescent dye is a fluorescent dye emitting fluorescence in long wavelength and near-infrared ray ranges.

Therefore, the present invention is directed to providing a novel two-photon absorbing fluorescent dye compound or a pharmaceutically acceptable salt thereof.

The present invention is also directed to providing a composition for cell or tissue imaging, which includes the compound or a chemically acceptable salt thereof as an active ingredient.

However, technical problems to be solved in the present invention are not limited to the above-described problems, and other problems which are not described herein will be fully understood by those of ordinary skill in the art from the following description.

In one aspect, the present invention provides a Py⁺BC derivative compound represented by Formula 1 below and a precursor PyBC derivative compound thereof, an ArBC derivative compound represented by Formula 2 below, a BtBC derivative compound represented by Formula 3 below, a KetoBC derivative compound represented by Formula 4 below, or a pharmaceutically acceptable salt thereof.

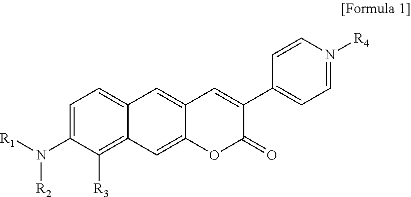

In Formula 1,

R₁ and R₂ are each independently hydrogen (H), a methyl group (Me), an allyl group, or a C₂-C₁₂ unsubstituted alkyl group;

R₁ is

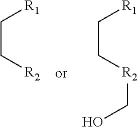

linked to R₂ in a ring;

R₃ is hydrogen (H), or

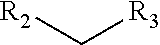

linked to R₂ in a ring;

R₄ is hydrogen (H), a methyl (Me) group, or oxygen (O).

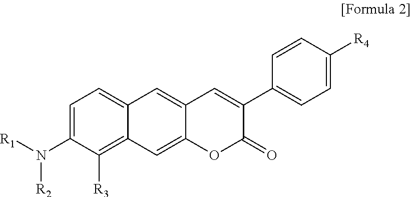

In Formula 2,

R₁ and R₂ are each independently hydrogen (H), a methyl group (Me), an allyl group, or a C₂-C₁₂ unsubstituted alkyl group;

R₁ is

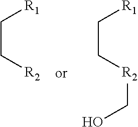

linked to R₂ in a ring;

R₃ is hydrogen (H), or

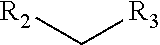

linked to R₂ in a ring;

R₄ is a hydroxyl group (OH), an amine group (NH₂), a nitro group (NO₂), an acetate group (OCOMe), or a carboxyl group (COOH).

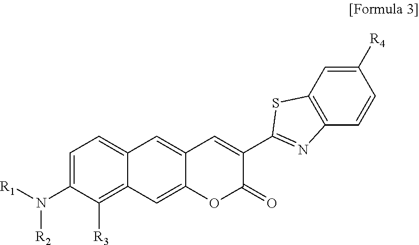

In Formula 3,

R₁ and R₂ are each independently hydrogen (H), a methyl group (Me), an allyl group, or a C₂-C₁₂ unsubstituted alkyl group;

R₁ is

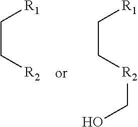

linked to R₂ in a ring;

R₃ is hydrogen (H), or

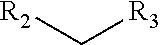

linked to R₂ in a ring; and

R₄ is a hydroxyl group (OH), an amine group (NH₂), a nitro group (NO₂), or a carboxyl group (COOH).

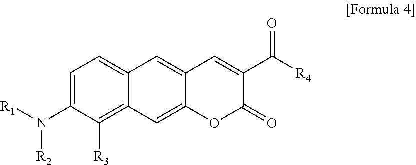

In Formula 4,

R₁ and R₂ are each independently hydrogen (H), a methyl group (Me), an allyl group, or a C₂-C₁₂ unsubstituted alkyl group;

R₁ is

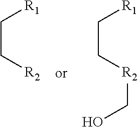

linked to R₂ in a ring;

R₃ is hydrogen (H), or

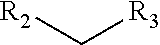

linked to R₂ in a ring;

R₄ is hydrogen (H), a methyl group (Me), an acetoxy group (COMe), or

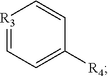

and

R₅ is hydrogen (H), a hydroxyl group (OH), an amine group (NH₂), or a nitro group (NO₂).

In one exemplary embodiment of the present invention, the compound may be a compound represented by one formula selected from the group consisting of Formulas 1g to 1k below.

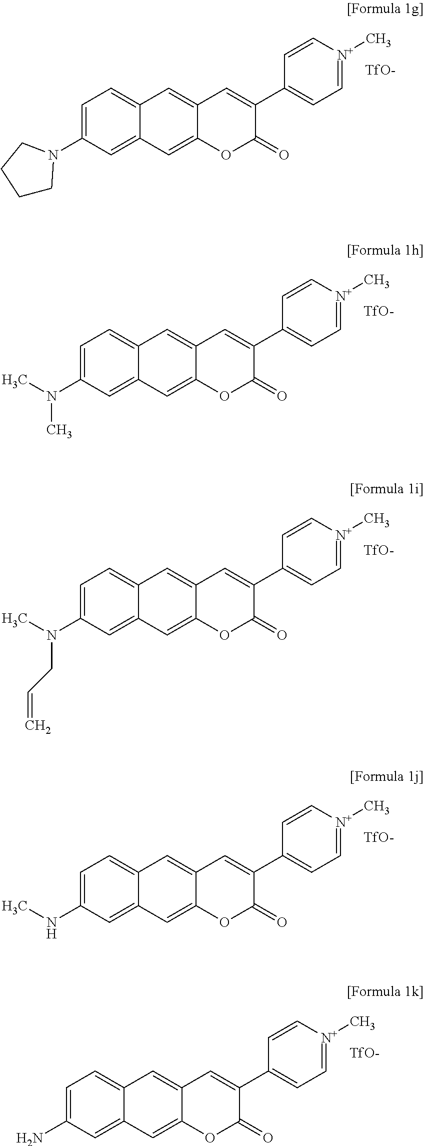

In another exemplary embodiment of the present invention, the compound may be a compound represented by Formula 1g.

In still another exemplary embodiment of the present invention, the compound may be a two-photon absorbing fluorescent dye.

In yet another exemplary embodiment of the present invention, the compound represented by one formula selected from the group consisting of Formulas 1g to 1k may be a two-photon absorbing near-infrared fluorescent dye.

In another aspect, the present invention provides a method and a composition for imaging cells or tissue using the compound or a pharmaceutically acceptable salt thereof.

In one exemplary embodiment of the present invention, the method may include treating cells or tissue in an animal model with the compound or a chemically acceptable salt thereof and examining the cells and tissue by fluorescence microscopy.

In another exemplary embodiment of the present invention, the compound may be a compound represented by Formula 1g.

In still another aspect, the present invention provides a method for preparing a compound represented by Formula 1a below from 3-hydroxy-6-(pyrrolidine-1-yl)-2-naphthaldehyde, which includes synthesizing 3-(pyridine-4-yl)-8-(pyrrolidine-1-yl)-2H-benzo[g]chromene-2-one by adding 4-pyridineacetic acid hydrochloride, triethylamine, 1-(3-dimethylaminopropyl)-3-ethylcarbodiimide, and 1-hydroxybenzotriazole hydrate.

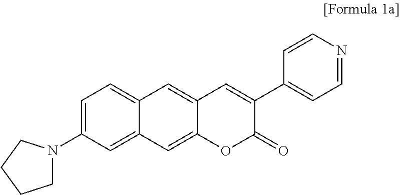 [SMILES: O=c1oc2cc3cc(N4CCCC4)ccc3cc2cc1-c1ccncc1]

In yet another aspect, the present invention provides a method for preparing a compound represented by Formula 1b below from 6-(dimethylamino)-3-hydroxy-2-naphthaldehyde, which includes synthesizing 8-(dimethylamino)-3-(pyridine-4-yl)-2H-benzo[g]chromene-2-one by adding 4-pyridineacetic acid hydrochloride, triethylamine, 1-(3-dimethylaminopropyl)-3-ethylcarbodiimide, and 1-hydroxybenzotriazole hydrate.

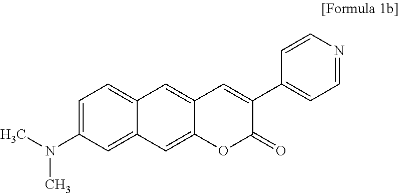 [SMILES: CN(C)c1ccc2cc3cc(-c4ccncc4)c(=O)oc3cc2c1]

In yet another aspect, the present invention provides a method for preparing a compound represented by Formula 1c below from 6-(allyl(methyl)amino)-3-hydroxy-2-naphthaldehyde, which includes synthesizing 8-(allyl(methyl)amino)-3-(pyridine-4-yl)-2H-benzo[g]chromene-2-one by adding 4-pyridineacetic acid hydrochloride, triethylamine, 1-(3-dimethylaminopropyl)-3-ethylcarbodiimide, and 1-hydroxybenzotriazole hydrate.

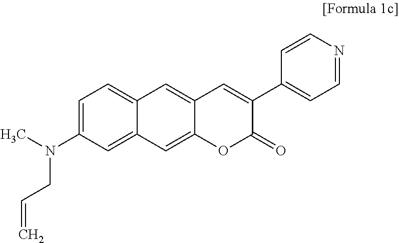 [SMILES: C=CCN(C)c1ccc2cc3cc(-c4ccncc4)c(=O)oc3cc2c1]

In yet another aspect, the present invention provides a method for preparing a compound represented by Formula 1d below from 3-hydroxy-6-(methylamino)-2-naphthaldehyde, which includes synthesizing 8-(methylamino)-3-(pyridine-4-yl)-2H-benzo[g]chromene-2-one by adding ethyl 4-pyridylacetate.

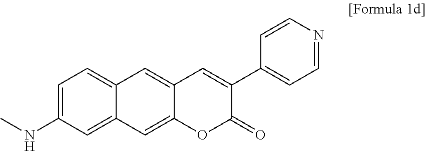 [SMILES: CNc1ccc2cc3cc(-c4ccncc4)c(=O)oc3cc2c1]

In yet another aspect, the present invention provides a method for preparing a compound represented by Formula 1e below from 6-(allylamino)-3-hydroxy-2-naphthaldehyde, which includes synthesizing 8-(allylamino)-3-(pyridine-4-yl)-2H-benzo[g]chromene-2-one by adding 4-pyridineacetic acid hydrochloride, triethylamine, 1-(3-dimethylaminopropyl)-3-ethylcarbodiimide, and 1-hydroxybenzotriazole hydrate.

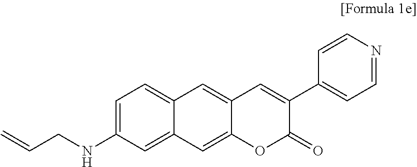 [SMILES: C=CCNc1ccc2cc3cc(-c4ccncc4)c(=O)oc3cc2c1]

In yet another aspect, the present invention provides a method for preparing a compound represented by Formula 1f below from 8-(allylamino)-3-(pyridine-4-yl)-2H-benzo[g]chromene-2-one, which includes synthesizing 8-amino-3-(pyridine-4-yl)-2H-benzo[g]chromene-2-one by adding tetrakis(triphenylphosphine)palladium and N, N′-dimethylbarbituric acid.

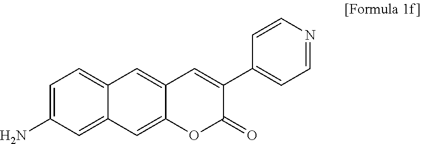 [SMILES: Nc1ccc2cc3cc(-c4ccncc4)c(=O)oc3cc2c1]

In yet another aspect, the present invention provides a method for preparing a compound represented by Formula 1g below from 3-(pyridine-4-yl)-8-(pyrrolidine-1-yl)-2H-benzo[g]chromene-2-one, which includes synthesizing 1-methyl-4-(2-oxo-8-(pyrrolidine-1-yl)-2H-benzo[g]chromene-3-yl)pyridinium trifluoromethanesulfonate by adding trifluoromethanesulfonate.

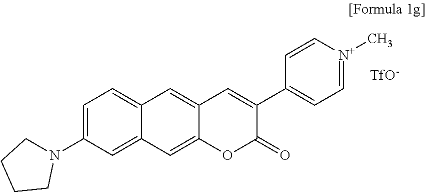

In yet another aspect, the present invention provides a method for preparing a compound represented by Formula 1h below from 8-(dimethylamino)-3-(pyridine-4-yl)-2H-benzo[g]chromene-2-one, which includes synthesizing 4-(8-(dimethylamino)-2-oxo-2H-benzo[g]chromene-3-yl)-1-methylpyridinium trifluoromethanesulfonate by adding trifluoromethanesulfonate.

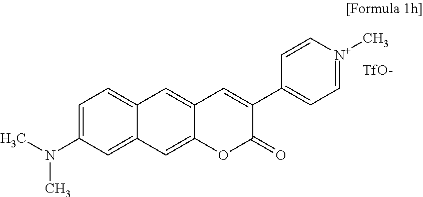

In yet another aspect, the present invention provides a method for preparing a compound represented by Formula 1i below from 8-(allyl(methyl)amino)-3-(pyridine-4-yl)-2H-benzo[g]chromene-2-one, which includes synthesizing 4-(8-(allyl(methyl)amino)-2-oxo-2H-benzo[g]chromene-3-yl)-1-methylpyridinium trifluoromethanesulfonate by adding trifluoromethanesulfonate.

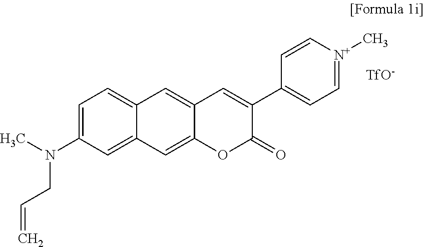

In yet another aspect, the present invention provides a method for preparing a compound represented by Formula 1j below from 8-(methylamino)-3-(pyridine-4-yl)-2H-benzo[g]chromene-2-one, which includes synthesizing 1-methyl-4-(8-(methylamino)-2-oxo-2H-benzo[g]chromene-3-yl)pyridinium trifluoromethanesulfonate by adding trifluoromethanesulfonate.

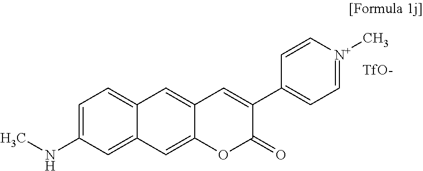

In yet another aspect, the present invention provides a method for preparing a compound represented by Formula 1k below from 8-amino-3-(pyridine-4-yl)-2H-benzo[g]chromene-2-one, which includes synthesizing 4-(8-amino-2-oxo-2H-benzo[g]chromene-3-yl)-1-methylpyridinium trifluoromethanesulfonate by adding trifluoromethanesulfonate.

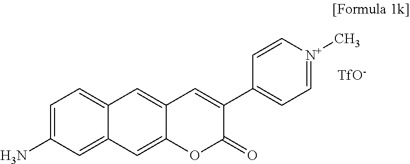

In yet another aspect, the present invention provides a method for preparing a compound represented by Formula 1l below from 6-(allyl(methyl)amino)-3-hydroxy-2-naphthaldehyde, which includes synthesizing 4-(8-(allyl(methyl)amino)-2-oxo-2H-benzo[g]chromene-3-yl)pyridine-1-oxide by adding ethyl 2-(pyridine-4-yl)acetate, meta-chloroperoxybenzoic acid, and 4-(2-ethoxy-2-oxo-ethyl)pyridine-1-oxide.

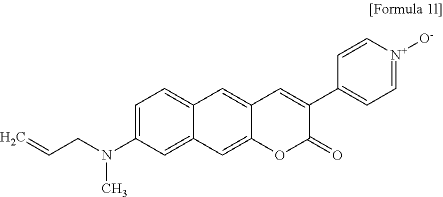 [SMILES: C=CCN(C)c1ccc2cc3cc(-c4cc[n+]([O-])cc4)c(=O)oc3cc2c1]

In yet another aspect, the present invention provides a method for preparing a compound represented by Formula 2a below from 6-(allylamino)-3-hydroxy-2-naphthaldehyde, which includes synthesizing 4-(8-(allylamino)-2-oxo-2H-benzo[g]chromene-3-yl)phenyl acetate by adding 4-acetoxybenzoic acid, triethylamine, 1-ethyl-3-(3-dimethylaminopropyl)-3-ethylcarbodiimide, and 1-hydroxybenzotriazole hydrate.

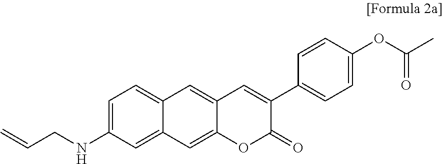 [SMILES: C=CCNc1ccc2cc3cc(-c4ccc(OC(C)=O)cc4)c(=O)oc3cc2c1]

In yet another aspect, the present invention provides a method for preparing a compound represented by Formula 2b below from 4-(8-(allylamino)-2-oxo-2H-benzo[g]chromene-3-yl)phenyl acetate, which includes synthesizing 8-(allylamino)-3-(4-hydroxyphenyl)-2H-benzo[g]chromene-2-one by adding sodium carbonate.

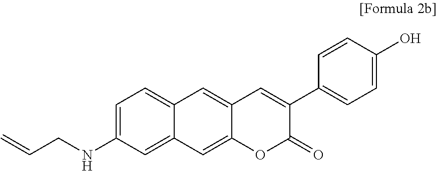 [SMILES: C=CCNc1ccc2cc3cc(-c4ccc(O)cc4)c(=O)oc3cc2c1]

In yet another aspect, the present invention provides a method for preparing a compound represented by Formula 2c below from 6-(allylamino)-3-hydroxy-2-naphthaldehyde, which includes synthesizing 8-(allylamino)-3-(4-nitrophenyl)-2H-benzo[g]chromene-2-one by adding 4-acetoxybenzoic acid, triethylamine, 1-(3-dimethylaminopropyl)-3-ethylcarbodiimide, and 1-hydroxybenzotriazole hydrate.

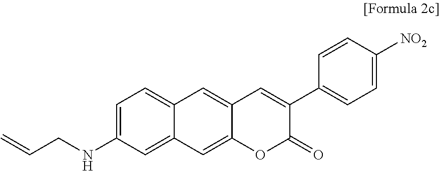 [SMILES: C=CCNc1ccc2cc3cc(-c4ccc([N+](=O)[O-])cc4)c(=O)oc3cc2c1]

In yet another aspect, the present invention provides a method for preparing a compound represented by Formula 2d below from 8-(allylamino)-3-(4-nitrophenyl)-2H-benzo[g]chromene-2-one, which includes synthesizing 8-(allylamino)-3-(4-aminophenyl)-2H-benzo[g]chromene-2-one by adding tin(II) chloride dihydrate.

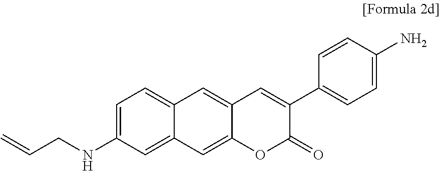 [SMILES: C=CCNc1ccc2cc3cc(-c4ccc(N)cc4)c(=O)oc3cc2c1]

In yet another aspect, the present invention provides a method for preparing a compound represented by Formula 3a from 6-(methylamino)-3-hydroxy-2-naphthaldehyde, which includes synthesizing 3-(benzo[d]thiazole-2-yl)-8-(methylamino)-2H-benzo[g]chromene-2-one by adding ethyl 2-(benzo[d]thiazole-2-yl)acetate and piperidine.

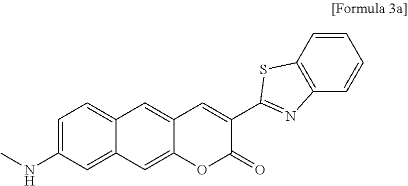 [SMILES: CNc1ccc2cc3cc(-c4nc5ccccc5s4)c(=O)oc3cc2c1]

In yet another aspect, the present invention provides a method for preparing a compound represented by Formula 3b below from 6-(allylamino)-3-hydroxy-2-naphthaldehyde, which includes synthesizing 8-(allylamino)-3-(benzo[d]thiazole-2-yl)-2H-benzo[g]chromene-2-one by adding ethyl 2-(benzo[d]thiazole-2-yl)acetate and piperidine.

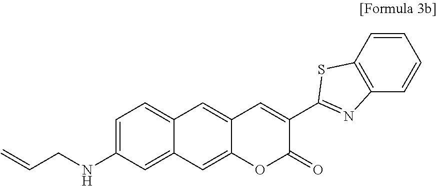 [SMILES: C=CCNc1ccc2cc3cc(-c4nc5ccccc5s4)c(=O)oc3cc2c1]

In yet another aspect, the present invention provides a method for preparing a compound represented by Formula 3c below from 3-hydroxy-6-(pyrrolidine-1-yl)-2-naphthaldehyde, which includes synthesizing 3-(benzo[d]thiazole-2-yl)-8-(pyrrolidine-1-yl)-2H-benzo[g]chromene-2-one by adding ethyl 2-(benzo[d]thiazole-2-yl)acetate and piperidine.

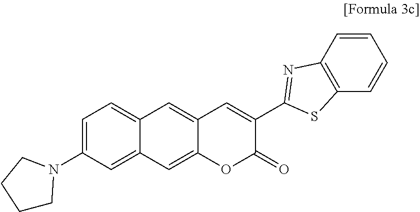 [SMILES: O=c1oc2cc3cc(N4CCCC4)ccc3cc2cc1-c1nc2ccccc2s1]

In yet another aspect, the present invention provides a method for preparing a compound represented by Formula 4a from 3-hydroxy-6-(methylamino)-2-naphthaldehyde, which includes synthesizing 3-acetyl-8-(methylamino)-2H-benzo[g]chromene-2-one by adding acetoacetate and piperidine.

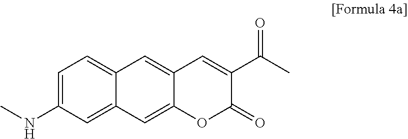 [SMILES: CNc1ccc2cc3cc(C(C)=O)c(=O)oc3cc2c1]

In yet another aspect, the present invention provides a method for preparing a compound represented by Formula 4b from 3-hydroxy-6-(pyrrolidine-1-yl)-2-naphthaldehyde, which includes synthesizing 3-acetyl-8-(methylamino)-2H-benzo[g]chromene-2-one by adding acetoacetate and piperidine.

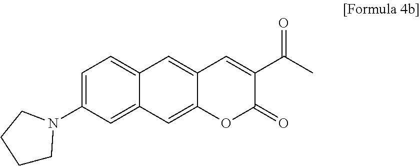 [SMILES: CC(=O)c1cc2cc3ccc(N4CCCC4)cc3cc2oc1=O]

In yet another aspect, the present invention provides a method for preparing a compound represented by Formula 4c from 3-hydroxy-6-(methylamino)-2-naphthaldehyde, which includes synthesizing 1-(8-(methylamino)-2-oxo-2H-benzo[g]chromene-3-yl)butane-1,3-dione by adding 4-hydroxy-6-methyl-2-pyrone and benzyltriethylammonium chloride.

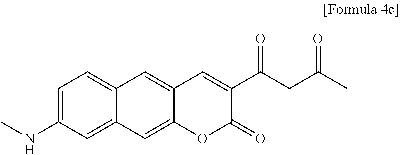 [SMILES: CNc1ccc2cc3cc(C(=O)CC(C)=O)c(=O)oc3cc2c1]

In yet another aspect, the present invention provides a method for preparing a compound represented by Formula 4d from 3-hydroxy-6-(2-(hydroxymethyl)pyrrolidine-1-yl)-2-naphthaldehyde, which includes synthesizing 1-(8-(2-(hydroxymethyl)pyrrolidine-1-yl)-2-oxo-2H-benzo[g]chromene-3-yl)butane-1,3-dione by adding 4-hydroxy-6-methyl-2-pyrone and benzyltriethylammonium chloride.

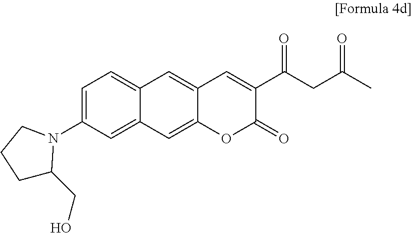 [SMILES: CC(=O)CC(=O)c1cc2cc3ccc(N4CCCC4CO)cc3cc2oc1=O]

In yet another aspect, the present invention provides a method for preparing a compound represented by Formula 4e from 6-(allylamino)-3-hydroxy-2-naphthaldehyde, which includes synthesizing 8-(allylamino)-3-benzoyl-2H-benzo[g]chromene-2-one by adding ethyl benzyl acetate and piperidine.

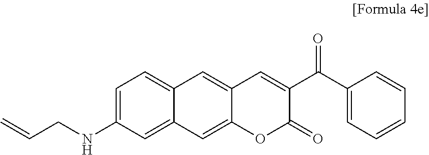 [SMILES: C=CCNc1ccc2cc3cc(C(=O)c4ccccc4)c(=O)oc3cc2c1]

In yet another aspect, the present invention provides a method for preparing a compound represented by Formula 4f from 6-(allylamino)-3-hydroxy-2-naphthaldehyde, which includes synthesizing 8-(allylamino)-3-(4-nitrobenzoyl)-2H-benzo[g]chromene-2-one by adding ethyl benzyl ethyl 3-(4-nitrophenyl)-3-oxopropanoate.

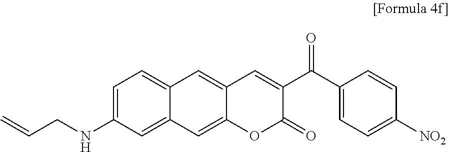 [SMILES: C=CCNc1ccc2cc3cc(C(=O)c4ccc([N+](=O)[O-])cc4)c(=O)oc3cc2c1]

In yet another aspect, the present invention provides a method for preparing a compound represented by Formula 4g from 8-(allylamino)-3-(4-nitrobenzoyl)-2H-benzo[g]chromene-2-one, which includes synthesizing 8-(allylamino)-3-(4-aminobenzoyl)-2H-benzo[g]chromene-2-one by adding tin(II) chloride dihydrate.

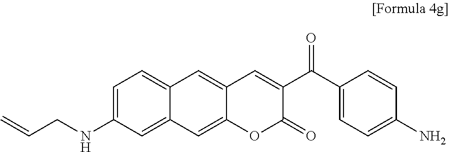 [SMILES: C=CCNc1ccc2cc3cc(C(=O)c4ccc(N)cc4)c(=O)oc3cc2c1]

In one exemplary embodiment of the present invention, the compound may be a compound represented by Formula 1g below.

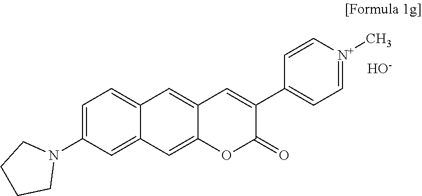

The Py⁺BC derivative compound of the present invention is a two-photon absorbing near-infrared fluorescent dye, which may minimize interference of autofluorescence compared to conventional two-photon absorbing fluorescent dyes such as acedan and naphthalimide, thereby obtaining a high-definition image, and therefore is expected to be effectively used in imaging studies. In addition, when the two-photon absorbing near-infrared fluorescent dye of the present invention is used, it is expected to be suitable for high resolution imaging of deep tissue.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 shows the wavelengths of maximum absorbance of Compounds 1c and 1g to 4g in a HEPES buffer, dioxane, ethanol, acetonitrile, dichloromethane, toluene, and a PBS buffer solution;

FIG. 2 shows the wavelengths of maximum emission of Compounds 1c and 1g to 4g in a HEPES buffer, dioxane, ethanol, acetonitrile, dichloromethane, toluene, and a PBS buffer solution; and

FIG. 3a shows two-photon fluorescence electron microscopy images of mouse kidney tissue treated with acedan, IminoPOS or Compound 1g at a concentration of 10 μM when being excited at 800 nm, 850 nm or 900 nm, respectively, FIG. 3b shows signal/noise (S/N) ratios according to the depth of tissue for mouse kidney tissue treated with acedan, IminoPOS, or Compound 1g at a concentration of 10 μM, and FIG. 3c shows two-photon fluorescence electron microscopy images of mouse kidney tissue treated with acedan, IminoPOS or Compound 1g at a concentration of 10 μM according to the depth of tissue (25 μm (surface), 125 μm (mid-tissue), or 175 μm (deep-tissue)).

### DETAILED DESCRIPTION OF EXEMPLARY EMBODIMENTS

The present invention provides a novel benzocoumarin-based two-photon absorbing fluorescent dye, for example, a Py⁺BC derivative compound represented by Formula 1 below and its precursor PyBC derivative compound, an ArBC derivative compound represented by Formula 2 below, a BtBC derivative compound represented by Formula 3 below, a KetoBC derivative compound represented by Formula 4 below, or a pharmaceutically acceptable salt thereof.

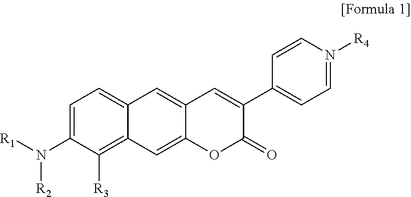

In Formula 1, R₁ and R₂ may be each independently hydrogen (H), a methyl group (Me), an allyl group, or a C₂-C₁₂ unsubstituted alkyl group;

R₁ may be

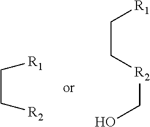

linked to R₂ in a ring;

R₃ may be hydrogen (H), or

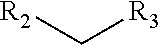

linked to R₂ in a ring; and

R₄ may be hydrogen (H), a methyl (Me) group, or oxygen (O), but the present invention is not limited thereto.

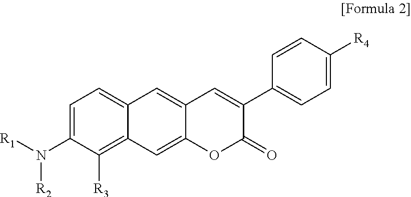

In Formula 2, R₁ and R₂ may be each independently hydrogen (H), a methyl group (Me), an allyl group, or a C₂-C₁₂ unsubstituted alkyl group;

R₁ may be

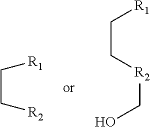

linked to R₂ in a ring;

R₃ may be hydrogen (H), or

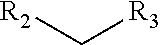

linked to R₂ in a ring; and

R₄ may be a hydroxyl group (OH), an amine group (NH₂), a nitro group (NO₂), an acetate group (OCOMe), or a carboxyl group (COOH), but the present invention is not limited thereto.

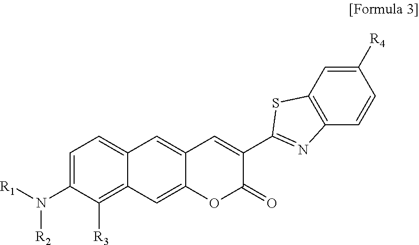

In Formula 3, R₁ and R₂ may be each independently hydrogen (H), a methyl group (Me), an allyl group, or a C₂-C₁₂ unsubstituted alkyl group;

R₁ may be

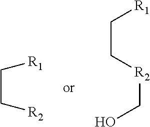

linked to R₂ in a ring;

R₃ may be hydrogen (H), or

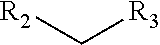

linked to R₂ in a ring; and

R₄ may be a hydroxyl group (OH), an amine group (NH₂), a nitro group (NO₂), or a carboxyl group (COOH), but the present invention is not limited thereto.

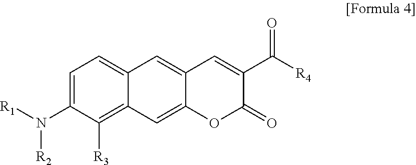

In Formula 4, R₁ and R₂ may be each independently hydrogen (H), a methyl group (Me), an allyl group, or a C₂-C₁₂ unsubstituted alkyl group;

R₁ may be

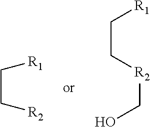

linked to R₂ in a ring;

R₃ may be hydrogen (H), or

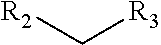

linked to R₂ in a ring;

R₄ may be hydrogen (H), a methyl group (Me), an acetoxy group (COMe), or

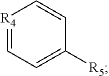

and

R₅ may be hydrogen (H), a hydroxyl group (OH), an amine group (NH₂), or a nitro group (NO₂), but the present invention is not limited thereto.

In one exemplary embodiment of the present invention, as a result of treatment of cells or tissue of an animal model with a representative one such as Compound 1g of the Py⁺BC derivative compounds of the present invention, compared to a conventional two-photon absorbing fluorescent dye, it was confirmed that a superior fluorescence image was able to be provided (refer to Example 29).

Therefore, the present invention may provide a composition for cell or tissue imaging, which includes the compound of the present invention or a chemically acceptable salt thereof as an active ingredient.

Hereinafter, to help in understanding the present invention, exemplary embodiments will be disclosed. However, the following examples are merely provided to more easily understand the present invention, and the scope of the present invention is not limited to the examples.

### Example 1. Synthesis of Compound 1a; 3-(pyridine-4-yl)-8-(pyrrolidine-1-yl)-2H-benzo[g]chromene-2-one

A general synthesis pathway of Compound 1a is shown in Reaction Scheme 1 below.

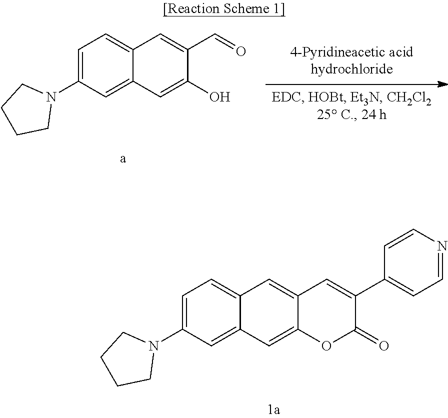 [SMILES: O=Cc1cc2ccc(N3CCCC3)cc2cc1O ; "4-Pyridineacetic acidhydrochloride" ; "EDC, HOBt, Et3N, CH2Cl225° C., 24 h" ; O=c1oc2cc3cc(N4CCCC4)ccc3cc2cc1-c1ccncc1]

The inventors synthesized Compound 1a, that is, 3-(pyridine-4-yl)-8-(pyrrolidine-1-yl)-2H-benzo[g]chromene-2-one.

Specifically, a known synthesis starting material, that is, Compound a (100 mg, 0.41 mmol) and 4-pyridineacetic acid hydrochloride (108 mg, 0.62 mmol) were dissolved in dichloromethane (2 mL), and then triethylamine (173 μL, 1.24 mmol) was added thereto. The resulting mixture was stirred for 10 minutes, and 1-(3-dimethylaminopropyl)-3-ethylcarbodiimide (EDC; 119 mg, 0.62 mmol) and 1-hydroxybenzotriazole hydrate (HOBt; 84 mg, 0.62 mmol) were added thereto. The resulting mixture was stirred at room temperature for 24 hours and extracted with dichloromethane, and then an organic layer was dried with anhydrous sodium sulfate. A crude product was purified by column chromatography in which it was passed through silica gel (eluent: MeOH/CH₂Cl₂=3/97), thereby obtaining an orange solid, Compound 1a (120 mg, 84%).

¹H NMR (CDCl₃, 300 MHz, 296 K): δ8.68 (dd, J=4.5, 1.8 Hz, 2H), 7.99 (s, 1H), 7.87 (s, 1H), 7.76 (d, J=9.3 Hz, 1H), 7.69 (dd, J=4.5, 1.8 Hz, 2H), 7.44 (s, 1H), 7.01 (dd, J=9.0, 2.4 Hz, 1H), 6.69 (d, J=2.4 Hz, 1H), 3.47 (t, J=6.9 Hz, 4H), 2.142.07 (m, 4H). ¹³C NMR (CDCl₃, 600 MHz, 296 K): δ159.8, 150.6, 149.5, 147.2, 142.4, 141.5, 137.2, 129.4, 128.5, 122.9, 122.1, 121.6, 115.8, 114.5, 108.7, 102.7, 47.2 (2 carbons), 25.0 (2 carbons). HRMS: m/z calcd for C₂₂H₁₈N₂O₂, 342.1368; found, 343.1447.

### Example 2. Synthesis of Compound 1b; 8-(dimethylamino)-3-(pyridine-4-yl)-2H-benzo[g]chromene-2-one

A general synthesis pathway of Compound 1b is shown in Reaction Scheme 2 below.

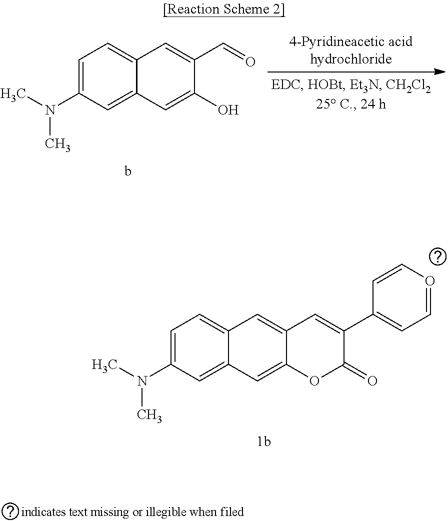

The inventors synthesized Compound 1b, that is, 8-(dimethylamino)-3-(pyridine-4-yl)-2H-benzo[g]chromene-2-one.

Specifically, a known synthesis starting material, that is, Compound b (30 mg, 0.14 mmol) and 4-pyridineacetic acid hydrochloride (108 mg, 0.62 mmol) were dissolved in dichloromethane (2 mL), and then triethylamine (173 μL, 1.24 mmol) was added thereto. The resulting mixture was stirred for 10 minutes, and 1-(3-dimethylaminopropyl)-3-ethylcarbodiimide (EDC; 119 mg, 0.62 mmol) and 1-hydroxybenzotriazole hydrate (HOBt; 84 mg, 0.62 mmol) were added thereto. The resulting mixture was stirred at room temperature for 24 hours and extracted with dichloromethane, and then an organic layer was dried with anhydrous sodium sulfate. A crude product was purified by column chromatography in which it was passed through silica gel (eluent: MeOH/CH₂Cl₂=3/97), thereby obtaining an orange solid, Compound 1b (33.4 mg, 74%).

¹H NMR (CDCl₃, 500 MHz, 296 K): δ8.69 (dd, J=4.5, 1.5 Hz, 2H), 8.01 (s, 1H), 7.90 (s, 1H), 7.77 (d, J=9.5 Hz, 1H), 7.69 (dd, J=4.5, 1.5 Hz, 2H), 7.48 (s, 1H), 7.16 (dd, J=9.0 2.5 Hz, 1H), 6.84 (d, J=2.0 Hz, 1H), 3.15 (s, 6H). ¹³C NMR (CDCl₃+MeOD, 600 MHz, 296 K): δ160.4, 151.0, 150.3, 149.5, 143.3, 142.4, 137.5, 129.8, 129.0, 123.7, 122.9, 122.1, 116.2, 115.4, 109.7, 104.1, 40.2 (2 carbons). HRMS: m/z calcd for C₂₀H₁₆N₂O₂, 316.1212; found, 317.1290.

### Example 3. Synthesis of Compound 1c; 8-(allyl(methyl)amino)-3-(pyridine-4-yl)-2H-benzo[g]chromene-2-one

A general synthesis pathway of Compound 1c is shown in Reaction Scheme 3 below.

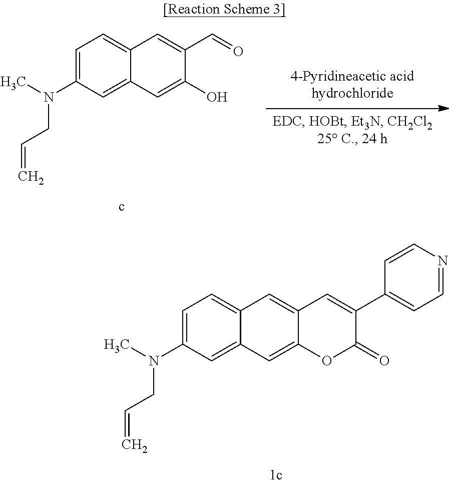 [SMILES: C=CCN(C)c1ccc2cc(C=O)c(O)cc2c1 ; "4-Pyridineacetic acidhydrochloride" ; "EDC, HOBt, Et3N, CH2Cl225° C., 24 h" ; C=CCN(C)c1ccc2cc3cc(-c4ccncc4)c(=O)oc3cc2c1]

The inventors synthesized Compound 1c, that is, 8-(allyl(methyl)amino)-3-(pyridine-4-yl)-2H-benzo[g]chromene-2-one.

Specifically, a known synthesis starting material, that is, Compound c (130 mg, 0.54 mmol) and 4-pyridineacetic acid hydrochloride (108 mg, 0.62 mmol) were dissolved in dichloromethane (2 mL), and then triethylamine (173 μL, 1.24 mmol) was added thereto. The resulting mixture was stirred for 10 minutes, and 1-(3-dimethylaminopropyl)-3-ethylcarbodiimide (EDC; 119 mg, 0.62 mmol) and 1-hydroxybenzotriazole hydrate (HOBt; 84 mg, 0.62 mmol) were added thereto. The resulting mixture was stirred at room temperature for 24 hours and extracted with dichloromethane, and then an organic layer was dried with anhydrous sodium sulfate. A crude product was purified by column chromatography in which it was passed through silica gel (eluent: MeOH/CH₂Cl₂=3/97), thereby obtaining an orange solid, Compound 1c (144 mg, 78%).

¹H NMR (CDCl₃, 300 MHz, 296 K): δ8.69 (d, J=5.4 Hz, 2H), 8.00 (s, 1H), 7.89 (s, 1H), 7.76 (d, J=9.3 Hz, 1H), 7.69 (dd, J=4.5, 1.5 Hz, 2H), 7.47 (s, 1H), 7.12 (dd, J=9.2, 2.4 Hz, 1H), 6.85 (d, J=2.4 Hz, 1H), 5.965.84 (m, 1H), 5.245.16 (m, 2H), 4.10 (dd, J=3 1.5 Hz, 2H), 3.13 (s, 3H). ¹³C NMR (CDCl₃, 300 MHz, 296 K): δ160.6, 150.9, 149.5, 149.2, 143.6, 142.7, 137.6, 132.7, 130.0, 129.1, 123.7, 123.0, 121.6, 116.5, 116.3, 115.3, 109.6, 103.9, 54.8, 49.548.4, 38.2. HRMS: m/z calcd for C₂₂H₁₈N₂O₂, 342.1368; found, 343.1447.

### Example 4. Synthesis of Compound 1d; 8-(methylamino)-3-(pyridine-4-yl)-2H-benzo[g]chromene-2-one

A general synthesis pathway of Compound 1d is shown in Reaction Scheme 4 below.

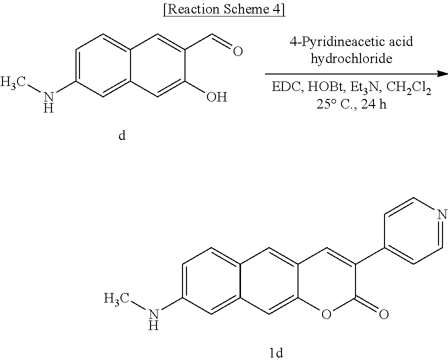 [SMILES: CNc1ccc2cc(C=O)c(O)cc2c1 ; "4-Pyridineacetic acidhydrochloride" ; "EDC, HOBt, Et3N, CH2Cl225° C., 24 h" ; CNc1ccc2cc3cc(-c4ccncc4)c(=O)oc3cc2c1]

The inventors synthesized Compound 1d, that is, 8-(methylamino)-3-(pyridine-4-yl)-2H-benzo[g]chromene-2-one.

Specifically, a known synthesis starting material, that is, Compound d (50 mg, 0.25 mmol) and ethyl 4-pyridylacetate (45.6 μL, 0.30 mmol) were dissolved in ethanol (2.5 mL), and two drops of piperidine were added to the produced solution, followed by stirring the resulting solution at 70° C. for 8 hours. After cooling at room temperature, the reaction mixture was concentrated under reduced pressure. The residue was treated and purified with a mixture of methanol and hexane (1:9), thereby obtaining an orange solid, Compound 1d (57 mg, 76%).

¹H NMR (DMSO, 300 MHz, 296 K): δ 8.65 (dd, J=4.8, 1.5 Hz, 2H), 8.49 (s, 1H), 8.11 (s, 1H), 7.78 (dd, J=9.0, 1.8 Hz, 2H), 7.51 (s, 1H), 6.99 (dd, J=9.0, 2.1 Hz, 1H), 6.68 (dd, J=14.7, 1.8 Hz, 2H), 2.82 (d, J=5.1 Hz, 3H). ¹³C NMR (DMSO-d₆, 600 MHz, 296 K): δ 160.0, 151.3, 150.6, 150.1, 143.5, 143.0, 138.4, 130.1, 130.0, 124.0, 123.1, 121.2, 119.3, 115.0, 108.8, 100.6. 29.9. HRMS: m/z calcd for C₁₉H₁₄N₂O₂, 302.1060; found, 303.1134.

### Example 5. Synthesis of Compound 1e; 8-(allylamino)-3-(pyridine-4-yl)-2H-benzo[g]chromene-2-one

A general synthesis pathway of Compound 1e is shown in Reaction Scheme 5 below.

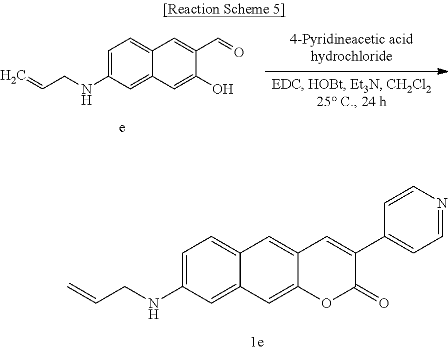 [SMILES: C=CCNc1ccc2cc(C=O)c(O)cc2c1 ; "4-Pyridineacetic acidhydrochloride" ; "EDC, HOBt, Et3N, CH2Cl225° C., 24 h" ; C=CCNc1ccc2cc3cc(-c4ccncc4)c(=O)oc3cc2c1]

The inventors synthesized Compound 1e, that is, 8-(allylamino)-3-(pyridine-4-yl)-2H-benzo[g]chromene-2-one.

Specifically, a known synthesis starting material, that is, Compound e (130 mg, 0.54 mmol) and 4-pyridineacetic acid hydrochloride (108 mg, 0.62 mmol) were dissolved in dichloromethane (2 mL), and then triethylamine (173 μL, 1.24 mmol) was added thereto. The resulting mixture was stirred for 10 minutes, and 1-(3-dimethylaminopropyl)-3-ethylcarbodiimide (EDC; 119 mg, 0.62 mmol) and 1-hydroxybenzotriazole hydrate (HOBt; 84 mg, 0.62 mmol) were added thereto. The resulting mixture was stirred at room temperature for 24 hours and extracted with dichloromethane, and then an organic layer was dried with anhydrous sodium sulfate. A crude product was purified by column chromatography in which it was passed through silica gel (eluent: MeOH/CH₂Cl₂=3/97), thereby obtaining an orange solid, Compound 1e (120 mg, 80%).

¹H NMR (CDCl₃, 300 MHz, 296 K): δ 8.55 (d, J=6 Hz, 2H), 8.00 (s, 1H), 7.84 (s, 1H), 7.68 (dd, J=4.8 1.0 Hz, 2H), 7.64 (d, J=9.0 Hz, 1H), 7.38 (s, 1H), 6.88 (dd, J=9.0 2.0 Hz, 1H), 6.69 (d, J=1.5 Hz, 1H), 5.985.90 (m, 1H), 5.315.17 (m, 2H), 3.86 (d, J=5.0 Hz, 2H), 3.64 (s, 3H). ¹³C NMR (CDCl₃, 500 MHz, 296 K): δ 160.6, 150.9, 149.2, 148.4, 143.5, 142.7, 138.0, 134.1, 129.8, 129.1, 124.5, 123.0, 121.9, 118.7, 116.6, 115.2, 109.6, 102.4, 49.448.4, 45.7.

### Example 6. Synthesis of Compound 1f; 8-amino-3-(pyridine-4-yl)-2H-benzo[g]chromene-2-one

A general synthesis pathway of Compound 1f is shown in Reaction Scheme 6 below.

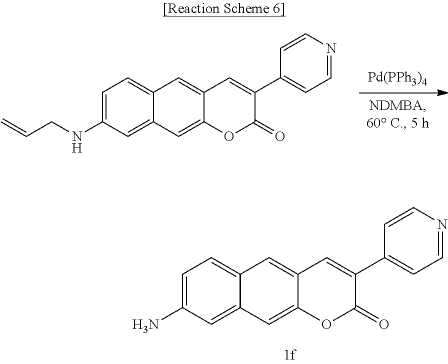 [SMILES: C=CCNc1ccc2cc3cc(-c4ccncc4)c(=O)oc3cc2c1 ; "NDMBA,60° C., 5 h" ; Nc1ccc2cc3cc(-c4ccncc4)c(=O)oc3cc2c1 ; "Pd(PPh3)4"]

The inventors synthesized Compound 1f, 8-amino-3-(pyridine-4-yl)-2H-benzo[g]chromene-2-one.

Specifically, Compound 1e (50 mg, 0.15 mmol) synthesized in Example 5 was dissolved in anhydrous methanol (2 mL), and tetrakis(triphenylphosphine)palladium (17.6 mg, 0.015 mmol) and N, N′-dimethylbarbituric acid (71.2 mg, 0.46 mmol) were added to the resulting solution under an argon gas. The mixture was stirred at 60° C. for 5 hours. The reaction mixture was concentrated under reduced pressure. A crude product was purified by column chromatography in which it was passed through silica gel (eluent: MeOH/CH₂Cl₂=1/9), thereby obtaining an orange solid, Compound 1f (28 mg, 64%).

¹H NMR (DMSO-d₆, 500 MHz, 296 K): δ 8.66 (d, J=3.6 Hz, 2H), 8.49 (s, 1H), 8.11 (s, 1H), 7.79 (d, J=6.6 Hz, 2H), 7.77 (s, 1H), 7.43 (s, 1H), 6.99 (dd, J=8.7, 2.4 Hz, 1H), 6.84 (d, J=1.8 Hz, 1H), 6.04 (s, 2H). ¹³C NMR (DMSO-d₆, 500 MHz, 296 K): δ 159.5, 150.6, 149.7, 149.6, 143.0, 137.5, 130.1, 129.6, 123.3, 122.5, 118.8, 114.5, 107.8, 104.0. HRMS: m/z calcd for C₁₈H₁₂N₂O₂, 288.0899; found, 289.0977.

### Example 7. Synthesis of Compound 1g; 1-methyl-4-(2-oxo-8-(pyrrolidine-1-yl)-2H-benzo[g]chromene-3-yl)pyridinium trifluoromethanesulfonate

A general synthesis pathway of Compound 1g is shown in Reaction Scheme 7 below.

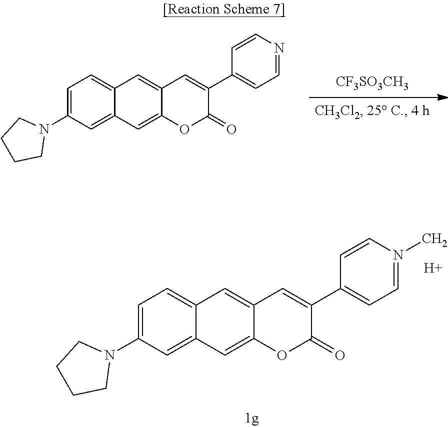

The inventors synthesized Compound 1g, that is, 1-methyl-4-(2-oxo-8-(pyrrolidine-1-yl)-2H-benzo[g]chromene-3-yl)pyridinium trifluoromethanesulfonate.

Specifically, trifluoromethanesulfonate (MeOTf; 25 μL, 0.22 mmol) was added dropwise to a solution of Compound 1a (50 mg, 0.15 mmol) synthesized in Example 1 in dichloromethane (1 mL), and the resulting solution was stirred at room temperature for 4 hours. The organic solvent was removed under reduced pressure, and then the residue was washed with dichloromethane and hexane (1:9) several times and vacuum-dried several times, thereby obtaining a red-violet solid, Compound 1g (63 mg, 85%).

¹H NMR (CDCl₃+MeOD, 300 MHz, 296 K): δ 8.508.48 (m, 3H), 8.36 (d, J=6.9 Hz, 2H), 7.87 (s, 1H), 7.58 (d, J=9.0 Hz, 1H), 7.15 (s, 1H), 6.82 (dd, J=9.2, 2.1 Hz, 1H), 6.47 (d, J=1.8 Hz, 1H), 4.12 (s, 3H), 3.27 (t, J=6.3 Hz, 4H), 1.891.85 (m, 4H). ¹³C NMR (DMSO-d₆, 500 MHz, 298 K): δ 158.9, 150.7, 150.0 148.1, 146.6, 144.7, 138.3, 131.6, 130.8, 124.6, 123.0, 116.7, 115.9, 114.2, 108.1, 103.0, 47.5, 47.1 (2 carbons), 24.9 (2 carbons). HRMS: m/z calcd for C₂₃H₂₁N₂O₂, 357.1603; found, 357.1603.

### Example 8. Synthesis of Compound 1h; 4-(8-(dimethylamino)-2-oxo-2H-benzo[g]chromene-3-yl)-1-methylpyridinium trifluoromethanesulfonate

A general synthesis pathway of Compound 1h is shown in Reaction Scheme 8 below.

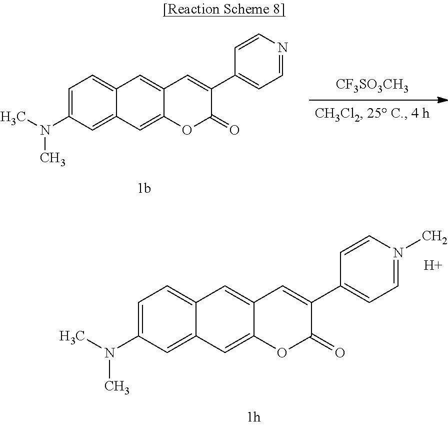

The inventors synthesized Compound 1h, that is, 4-(8-(dimethylamino)-2-oxo-2H-benzo[g]chromene-3-yl)-1-methylpyridinium trifluoromethanesulfonate.

Specifically, trifluoromethanesulfonate (MeOTf; 25 μL, 0.22 mmol) was added dropwise to a solution of Compound 1b (30 mg, 0.095 mmol) synthesized in Example 2 in dichloromethane (1 mL), and the resulting solution was stirred at room temperature for 4 hours. The organic solvent was removed under reduced pressure, and then the residue was washed with dichloromethane and hexane (1:9) several times and vacuum-dried several times, thereby obtaining a red-violet solid, Compound 1h (37 mg, 81%).

¹H NMR (MeOD+CDCl₃, 300 MHz, 296 K): δ 8.808.78 (m, 3H), 8.61 (d, J=7.2 Hz, 2H), 8.16 (s, 1H), 7.85 (d, J=9.0 Hz, 1H), 7.46 (s, 1H), 7.24 (dd, J=9.3, 2.4 Hz, 1H), 4.37 (s, 3H), 3.18 (s, 6H). ¹³C NMR (DMSO-d₆, 500 MHz, 296 K): δ 159.0, 150.7, 150.7, 150.2, 146.7, 144.9, 138.1, 131.3, 130.6, 130.1, 125.0, 123.1, 117.0, 116.4, 114.7, 108.7, 103.6, 47.2. HRMS: m/z calcd for C₂₁H₁₉N₂O₂, 331.1447; found, 331.1447.

### Example 9. Synthesis of Compound 1i; 4-(8-(allyl(methyl)amino)-2-oxo-2H-benzo[g]chromene-3-yl)-1-methylpyridinium trifluoromethanesulfonate

A general synthesis pathway of Compound 1i is shown in Reaction Scheme 9 below.

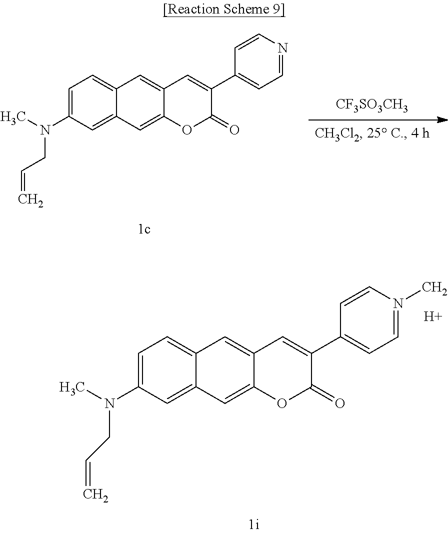

The inventors synthesized Compound 1i, that is, 4-(8-(allyl(methyl)amino)-2-oxo-2H-benzo[g]chromene-3-yl)-1-methylpyridinium trifluoromethanesulfonate.

Specifically, trifluoromethanesulfonate (MeOTf; 25 μL, 0.22 mmol) was added dropwise to a solution of Compound 1c (44 mg, 0.13 mmol) synthesized in Example 3 in dichloromethane (1 mL), and the resulting solution was stirred at room temperature for 4 hours. The organic solvent was removed under reduced pressure, and then the residue was washed with dichloromethane and hexane (1:9) several times and vacuum-dried several times, thereby obtaining a red-violet solid, Compound 1i (58 mg, 88%).

¹H NMR (CDCl₃+MeOD, 300 MHz, 296 K): δ 8.638.58 (m, 3H), 8.49 (d, J=6.9 Hz, 2H), 8.02 (s, 1H), 7.71 (d, J=9.3 Hz, 1H), 7.31 (s, 1H), 7.04 (dd, J=9.2, 2.7 Hz, 1H), 6.73 (d, J=2.4 Hz, 1H), 5.845.72 (m, 1H), 5.125.03 (m, 2H), 4.26 (s, 3H), 4.02 (d, J=4.5 Hz, 2H), 3.05 (s, 3H). ¹³C NMR (DMSO-d₆, 500 MHz, 296 K): δ 159.0, 150.6, 150.1, 149.7, 146.7, 144.8, 138.2, 133.3, 131.3, 125.0, 123.2, 122.0, 119.4, 116.9, 116.4, 116.3, 114.7, 108.7, 103.7, 53.9, 47.2, 38.1. HRMS: m/z calcd for C₂₃H₂₁N₂O₂, 357.1603; found, 357.1603.

### Example 10. Synthesis of Compound 1j; 1-methyl-4-(8-(methylamino)-2-oxo-2H-benzo[g]chromene-3-yl)pyridinium trifluoromethanesulfonate

A general synthesis pathway of Compound 1j is shown in Reaction Scheme 10 below.

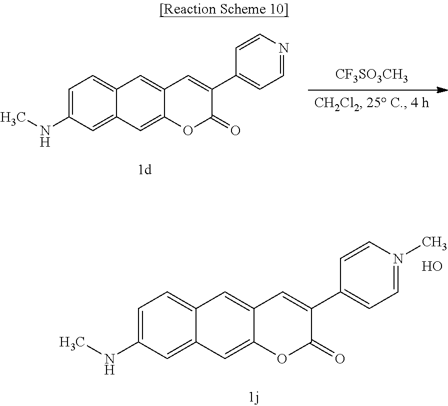

The inventors synthesized Compound 1j, that is, 1-methyl-4-(8-(methylamino)-2-oxo-2H-benzo[g]chromene-3-yl)pyridinium trifluoromethanesulfonate.

Specifically, trifluoromethanesulfonate (MeOTf; 25 μL, 0.22 mmol) was added dropwise to a solution of Compound 1d (40 mg, 0.13 mmol) synthesized in Example 4 in dichloromethane (1 mL), and the resulting solution was stirred at room temperature for 4 hours. The organic solvent was removed under reduced pressure, and then the residue was washed with dichloromethane and hexane (1:9) several times and vacuum-dried several times, thereby obtaining a red solid, Compound 1j (52 mg, 84%).

¹H NMR (DMSO-d₆, 300 MHz, 296 K): δ 8.94 (d, J=6.9 Hz, 2H), 8.85 (s, 1H), 8.51 (d, J=7.2 Hz, 2H), 8.13 (s, 1H), 7.79 (d, J=9.0 Hz, 1H), 7.45 (s, 1H), 6.99 (dd, J=9.2, 2.1 Hz, 1H), 6.69 (d, J=1.8 Hz, 1H), 4.32 (s, 3H), 2.83 (s, 3H). ¹³C NMR (DMSO-d₆, 300 MHz, 296 K): δ 159.1, 150.9, 150.9, 150.2, 146.8, 144.8, 139.1, 131.3, 130.3, 124.9, 123.6, 119.1, 116.4, 114.1, 108.3, 100.2, 4701, 29.3. HRMS: calcd for C₂₀H₁₇N₂O₂, 317.1285; found, 317.1290.

### Example 11. Synthesis of Compound 1k; 4-(8-amino-2-oxo-2H-benzo[g]chromene-3-yl)-1-methylpyridinium trifluoromethanesulfonate

A general synthesis pathway of Compound 1k is shown in Reaction Scheme 11 below.

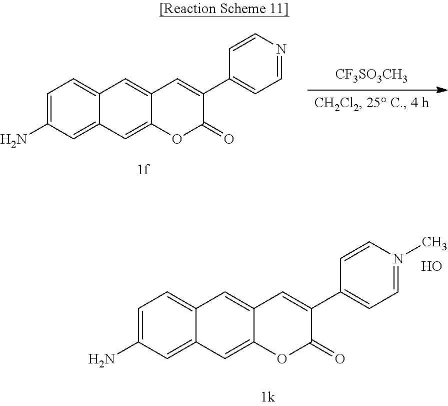

The inventors synthesized Compound 1k, that is, 4-(8-amino-2-oxo-2H-benzo[g]chromene-3-yl)-1-methylpyridinium trifluoromethanesulfonate.

Specifically, trifluoromethanesulfonate (MeOTf; 25 μL, 0.22 mmol) was added dropwise to a solution of Compound 1f (10 mg, 0.023 mmol) synthesized in Example 6 in dichloromethane (1 mL), and the resulting solution was stirred at room temperature for 4 hours. The organic solvent was removed under reduced pressure, and then the residue was washed with dichloromethane and hexane (1:9) several times and vacuum-dried several times, thereby obtaining a red solid, Compound 1k (7 mg, 67%).

¹H NMR (DMSO-d₆, 300 MHz, 296 K): δ 8.97 (d, J=6.6 Hz, 2H), 8.90 (s, 1H), 8.54 (d, J=6.6 Hz, 2H), 8.18 (s, 1H), 7.84 (d, J=9.3 Hz, 1H), 7.46 (s, 1H), 7.01 (dd, J=9.0 2.1 Hz, 1H), 6.84 (s, 1H), 6.33 (s, 2H), 4.32 (s, 3H). ¹³C NMR (DMSO-d₆, 500 MHz, 296 K): δ 159.1, 150.8, 150.8, 150.3, 146.9, 144.9, 138.7, 131.5, 131.0, 125.0, 123.5, 119.1, 116.7, 114.2, 107.9, 104.0, 47.2. HRMS: calcd for C₁₉H₁₅N₂O₂, 303.1133; found, 303.1134.

### Example 12. Synthesis of Compound 1l; 4-(8-(allyl(methyl)amino)-2-oxo-2H-benzo[g]chromene-3-yl)pyridine-1-oxide

A general synthesis pathway of Compound 1l is shown in Reaction Scheme 12 below.

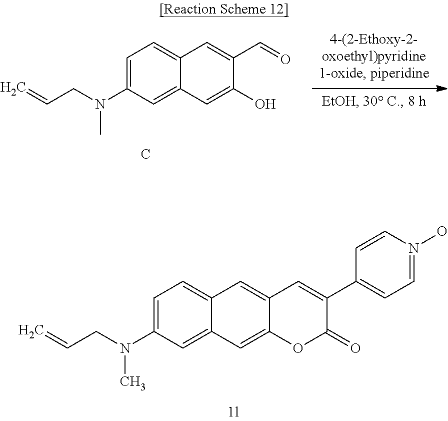

The inventors synthesized Compound 1l, that is, 4-(8-(allyl(methyl)amino)-2-oxo-2H-benzo[g]chromene-3-yl)pyridine-1-oxide.

Specifically, a solution of ethyl 2-(pyridine-4-yl)acetate (200 mg, 1.21 mmol) and meta-chloroperoxybenzoic acid (mCPBA; 418 mg, 2.42 mmol) in tetrahydrofuran (THF; 2 mL) was stirred at room temperature for 10 hours. The resulting mixture was concentrated under reduced pressure, and a crude product was purified by column chromatography in which it was passed through silica gel (eluent: MeOH/CH₂Cl₂=7/93), thereby obtaining 4-(2-ethoxy-2-oxo-ethyl)pyridine 1-oxide (146 mg, 67%). An ethanol (1 mL) solution of known synthesis starting materials, that is, Compound c (50 mg, 0.21 mmol) and 4-(2-ethoxy-2-oxo-ethyl)pyridine 1-oxide (45 mg, 0.25 mmol) was treated with two drops of piperidine, and the resulting solution was stirred at 30° C. for 8 hours. The reaction mixture was concentrated under reduced pressure, and a crude product was purified by column chromatography in which it was passed through silica gel (eluent: MeOH/CH₂Cl₂=3/97), thereby obtaining an orange solid, Compound 1l (54 mg, 73%).

¹H NMR (CDCl₃, 300 MHz, 296 K): δ 8.24 (dd, J=5.4 2.1 Hz, 2H), 7.99 (s, 1H), 7.88 (s, 1H), 7.79 (dd, J=5.4 2.1 Hz, 2H), 7.74 (s, 1H), 7.45 (s, 1H), 7.12 (dd, J=9.2, 2.7 Hz, 1H), 6.84 (d, J=2.4 Hz, 1H), 5.955.83 (m, 1H), 5.245.15 (m, 2H), 4.10 (dd, J=2.7 1.8 Hz, 2H), 3.13 (s, 3H). ¹³C NMR (CDCl₃, 500 MHz, 296 K): δ160.4, 150.9, 149.7, 142.1, 138.8, 137.9, 135.2, 132.7, 130.2, 129.4, 125.3, 123.9, 119.7, 116.7, 116.5, 115.3, 109.8, 104.1, 54.9, 49.848.7, 38.4. HRMS: m/z calcd for C₂₂H₁₈N₂O₃, 358.1317; found, 359.1396.

### Example 13. Synthesis of Compound 2a; 4-(8-(allylamino)-2-oxo-2H-benzo[g]chromene-3-yl)phenyl acetate

A general synthesis pathway of Compound 2a is shown in Reaction Scheme 13 below.

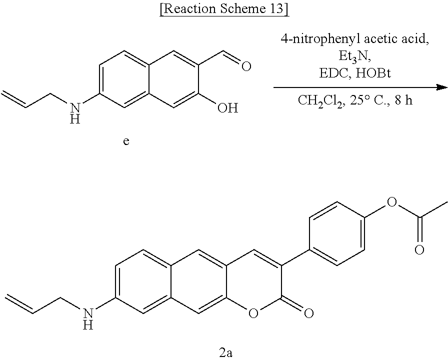

The inventors synthesized Compound 2a, that is, 4-(8-(allylamino)-2-oxo-2H-benzo[g]chromene-3-yl)phenyl acetate.

Specifically, a known synthesis starting material, that is, Compound e (100 mg, 0.41 mmol) was mixed with 4-acetoxybenzoic acid (120 mg, 0.62 mmol), triethylamine (289 μL, 2.07 mmol), 1-ethyl-3-(3-dimethylaminopropyl)carbodiimide (EDC; 96.5 mg, 0.62 mmol), and 1-hydroxybenzotriazole hydrate (HOBt; 95.2 mg, 0.62 mmol). The resulting mixture was stirred at room temperature for 8 hours and concentrated under reduced pressure, and a crude product was purified by column chromatography in which it was passed through silica gel (eluent: MeOH/CH₂Cl₂=3/97), thereby obtaining an orange solid, Compound 2a (126 mg, 80%).

¹H NMR (CDCl₃, 300 MHz, 296 K): δ 7.84 (d, 2H, J=5.7 Hz), 7.74 (d, 2H, J=6.6 Hz), 7.69 (d, 1H, J=9.0 Hz), 7.45 (s, 1H), 7.19 (s, 1H), 7.16 (s, 1H), 6.89 (d, 1H, J=9.0 Hz), 6.77 (d, 1H, J=2.1 Hz), 6.02 (m, 1H, J=38 Hz), 7.35 (d, 1H, J=17 Hz), 5.25 (d, 1H, J=11 Hz), 4.27 (t, 1H, J=11 Hz), 3.94 (d, 2H, J=3.6 Hz), 2.33 (s, 3H).

### Example 14. Synthesis of Compound 2b; 8-(allylamino)-3-(4-hydroxyphenyl)-2H-benzo[g]chromene-2-one

A general synthesis pathway of Compound 2b is shown in Reaction Scheme 14 below.

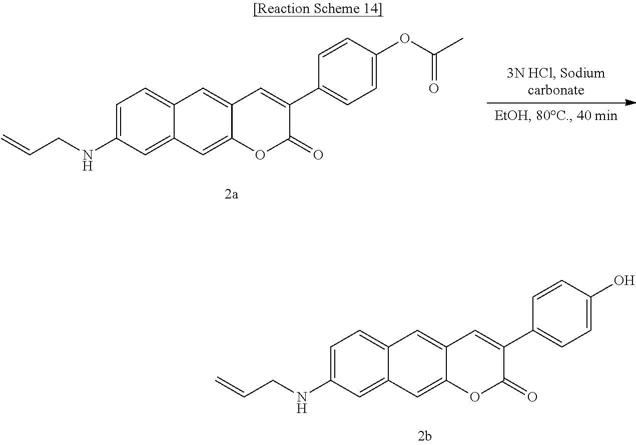

The inventors synthesized Compound 2b, that is, 8-(allylamino)-3-(4-hydroxyphenyl)-2H-benzo[g]chromene-2-one.

Specifically, Compound 2a (20 mg, 0.052 mmol) synthesized in Example 13 and 3N HCl (1 mL) were added to ethanol (2 mL). The resulting mixture was stirred at 80° C. for 40 minutes. Sodium carbonate was added dropwise to neutralize the resulting mixture. The reaction mixture was concentrated under reduced pressure, thereby obtaining an orange solid, Compound 2b (18 mg, 97%).

¹H NMR (MeOD, 300 MHz, 296 K): δ 7.91 (d, 2H, J=9.3 Hz), 7.60 (d, 2H, J=2.4 Hz), 7.51 (d, 1H, J=2.1 Hz), 7.35 (s, 1H), 6.95 (d, 1H, J=8.7 Hz), 6.78 (m, 3H), 5.92 (m, 1H), 5.26 (d, 1H, J=17 Hz), 5.12 (d, 1H, J=11 Hz), 3.83 (d, 2H, J=3.6 Hz).

### Example 15. Synthesis of Compound 2c; 8-(allylamino)-3-(4-nitrophenyl)-2H-benzo[g]chromene-2-one

A general synthesis pathway of Compound 2c is shown in Reaction Scheme 15 below.

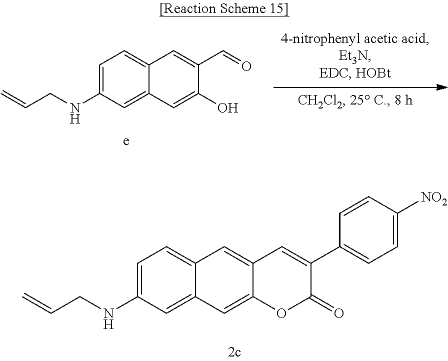

The inventors synthesized Compound 2c, that is, 8-(allylamino)-3-(4-nitrophenyl)-2H-benzo[g]chromene-2-one.

Specifically, 4-nitrophenyl acetic acid (113 mg, 0.62 mmol) and triethylamine (289 μL, 2.07 mmol) were added to dichloromethane (2 mL). The resulting mixture was stirred for 10 minutes, and known synthesis starting materials, that is, Compound e (100 mg, 0.41 mmol), 1-(3-dimethylaminopropyl)-3-ethylcarbodiimide (EDC; 96.5 mg, 0.62 mmol) and 1-hydroxybenzotriazole hydrate (HOBt; 95.2 mg, 0.62 mmol) were added. The mixture was stirred at room temperature for 8 hours and extracted with dichloromethane, and an organic layer was dried with anhydrous sodium sulfate. A crude product was purified by column chromatography in which it was passed through silica gel (eluent: MeOH/CH₂Cl₂=3/97), thereby obtaining an orange solid, Compound 2c (184 mg, 80%).

¹H NMR (DMSO, 300 bMHz, 296 bK): δ 8.47 (s, 1H), 8.31 (d, 2H, J=9.0 Hz), 8.12 (s, 1H), 8.04 (d, 2H, J=7.2 Hz), 7.78 (d, 1H, J=9.0 Hz), 7.48 (s, 1H), 7.06 (d, 1H, J=9.0 Hz), 6.83 (t, 1H, J=12 Hz), 6.76 (s, 1H), 6.00 (m, 1H, J=33 Hz), 5.30 (d, 1H, J=17 Hz), 5.17 (d, 1H, J=11 Hz), 3.86 (d, 2H, J=4.8 Hz).

### Example 16. Synthesis of Compound 2d; 8-(allylamino)-3-(4-aminophenyl)-2H-benzo[g]chromene-2-one

A general synthesis pathway of Compound 2d is shown in Reaction Scheme 16 below.

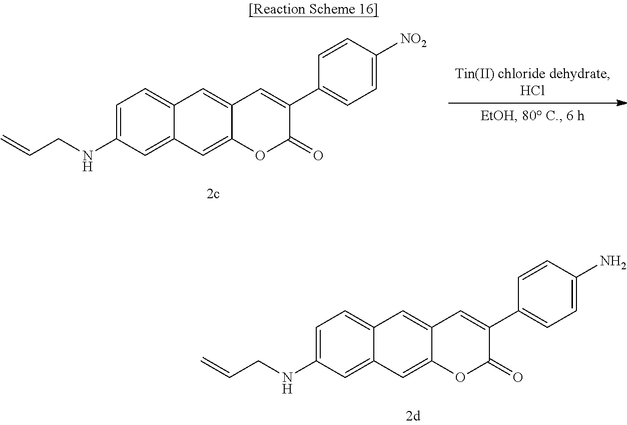 [SMILES: C=CCNc1ccc2cc3cc(-c4ccc([N+](=O)[O-])cc4)c(=O)oc3cc2c1 ; "EtOH, 80° C., 6 h" ; "Tin(II) chloride dehydrate,HCl" ; C=CCNc1ccc2cc3cc(-c4ccc(N)cc4)c(=O)oc3cc2c1]

The inventors synthesized Compound 2d, that is, 8-(allylamino)-3-(4-aminophenyl)-2H-benzo[g]chromene-2-one.

Specifically, Compound 2c (20 mg, 0.054 mmol) synthesized in Example 15 was dissolved in ethanol (2 mL), and tin(II) chloride dihydrate (77.6 mg, 0.34 mmol) and hydrochloric acid (0.12 mL) were added. The resulting mixture was stirred at 80° C. for 6 hours. The reaction mixture was neutralized by adding 10% sodium carbonate dropwise, and dried under reduced pressure, thereby obtaining an orange solid, Compound 2d (17.9 mg, 97%).

¹H NMR (DMSO, 300 bMHz, 296 bK): δ 8.01 (d, 2H, J=12 Hz), 7.71 (d, 1H, J=9.3 Hz), 7.45 (d, 2H, J=8.4 Hz), 7.41 (s, 1H), 7.02 (d, 1H, J=9.3 Hz), 6.72 (s, 1H), 6.61 (d, 2H, J=8.4 Hz), 6.00 (m, 1H, J=33 Hz), 5.30 (d, 1H, J=17 Hz), 5.16 (d, 1H, J=10 Hz), 3.82 (d, 2H, J=11 Hz).

### Example 17. Synthesis of Compound 3a; 3-(benzo[d]thiazole-2-yl)-8-(methylamino)-2H-benzo[g]chromene-2-one

A general synthesis pathway of Compound 3a is shown in Reaction Scheme 17 below.

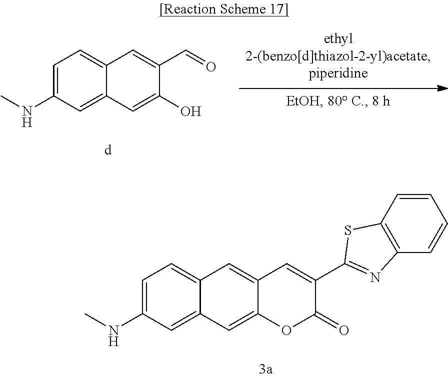

The inventors synthesized Compound 3a, that is, 3-(benzo[d]thiazole-2-yl)-8-(methylamino)-2H-benzo[g]chromene-2-one.

Specifically, a known synthesis starting material, that is, Compound d (20 mg, 0.1 mmol) and ethyl 2-(benzo[d]thiazole-2-yl)acetate (21 μL, 0.12 mmol) were dissolved in ethanol (2 mL), and then piperidine (1 drop) was added thereto. The reaction mixture was stirred at 80° C. for 8 hours. The reaction mixture was concentrated under reduced pressure, thereby obtaining an orange solid, Compound 3a (35 mg, 97%).

¹H NMR (CDCl₃, 300 MHz, 296 K): δ 9.18 (s, 1H), 8.08 (d, 1H, J=8.4 Hz), 8.02 (s, 1H), 7.98 (d, 1H, J=7.8 Hz), 7.72 (d, 1H, J=9 Hz), 7.52 (m, 2H, J=14 Hz), 7.41 (t, 1H, J=14 Hz), 6.89 (d, 1H, J=2.4 Hz), 6.74 (d, 1H, J=2.1 Hz), 3.00 (s, 1H).

### Example 18. Synthesis of Compound 3b; 8-(allylamino)-3-(benzo[d]thiazole-2-yl)-2H-benzo[g]chromene-2-one

A general synthesis pathway of Compound 3b is shown in Reaction Scheme 18 below.

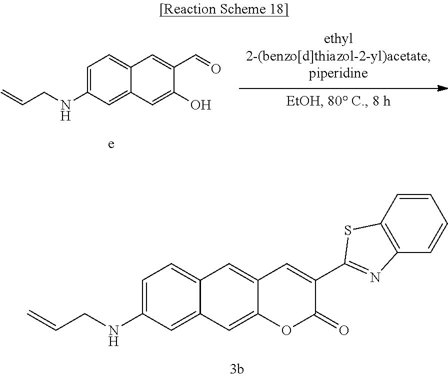

The inventors synthesized Compound 3b, that is, 8-(allylamino)-3-(benzo[d]thiazole-2-yl)-2H-benzo[g]chromene-2-one.

Specifically, a known synthesis starting material, that is, Compound e (50 mg, 0.22 mmol) and ethyl 2-(benzo[d]thiazole-2-yl)acetate (21 μL, 0.12 mmol) were dissolved in ethanol (2 mL), and then piperidine (1 drop) was added thereto. The reaction mixture was stirred at 80° C. for 8 hours. The reaction mixture was concentrated under reduced pressure, thereby obtaining an orange solid, Compound 3b (82 mg, 97%).

¹H NMR (DMSO, 300 MHz, 296 K): δ 9.18 (s, 1H), 8.37 (s, 1H), 8.16 (d, 1H, J=7.8 Hz), 8.07 (d, 1H, J=8.1 Hz), 7.78 (d, 1H, J=9 Hz), 7.56 (d, 1H, J=7.2 Hz), 7.53 (s, 1H), 7.46 (t, 1H, J=15.9 Hz), 7.08 (d, 1H, J=9.3 Hz), 7.02 (t, 1H, J=7.8 Hz), 6.78 (s, 1H), 6.01 (m, 1H, J=38 Hz), 5.30 (d, 1H, J=17 Hz), 5.17 (d, 1H, J=10 Hz), 3.87 (t, 2H, J=11 Hz).

### Example 19. Synthesis of Compound 3c; 3-(benzo[d]thiazole-2-yl)-8-(pyrrolidine-1-yl)-2H-benzo[g]chromene-2-one

A general synthesis pathway of Compound 3c is shown in Reaction Scheme 19 below.

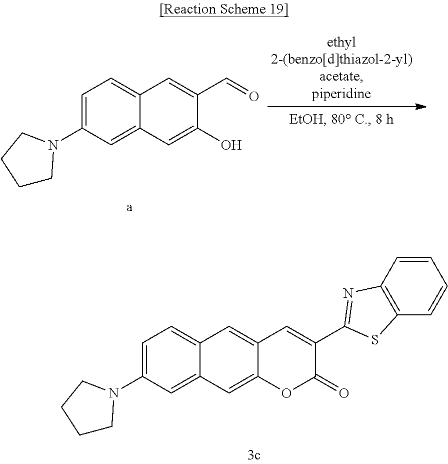 [SMILES: O=Cc1cc2ccc(N3CCCC3)cc2cc1O ; O=c1oc2cc3cc(N4CCCC4)ccc3cc2cc1-c1nc2ccccc2s1]

The inventors synthesized Compound 3c, that is, 3-(benzo[d]thiazole-2-yl)-8-(pyrrolidine-1-yl)-2H-benzo[g]chromene-2-one.

Specifically, a known synthesis starting material, that is, Compound a (50 mg, 0.21 mmol) and ethyl 2-(benzo[d]thiazole-2-yl)acetate (21 μL, 0.12 mmol) were dissolved in ethanol (2 mL), and then piperidine (1 drop) was added thereto. The reaction mixture was stirred at 80° C. for 8 hours. The reaction mixture was concentrated under reduced pressure, thereby obtaining an orange solid, Compound 3c (81 mg, 97%).

¹H NMR (CDCl₃, 300 MHz, 296 K): δ 9.04 (s, 1H), 8.07 (d, 1H, J=8.7 Hz), 7.96 (m, 2H, J=6.6 Hz), 7.77 (d, 1H, J=9.3 Hz), 7.51 (t, 1H, J=14 Hz), 7.44 (s, 1H), 7.42 (t, 1H, J=8.1 Hz), 6.98 (d, 1H, J=9 Hz), 6.67 (s, 1H), 3.44 (t, 4H, J=9 Hz), 2.06 (t, 4H, J=9 Hz).

### Example 20. Synthesis of Compound 4a; 3-acetyl-8-(methylamino)-2H-benzo[g]chromene-2-one

A general synthesis pathway of Compound 4a is shown in Reaction Scheme 20 below.

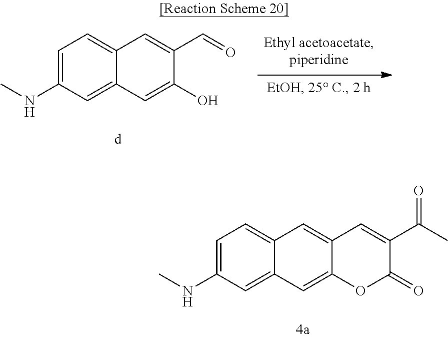 [SMILES: CNc1ccc2cc(C=O)c(O)cc2c1 ; "EtOH, 25° C., 2 h" ; "Ethyl acetoacetate,piperidine" ; CNc1ccc2cc3cc(C(C)=O)c(=O)oc3cc2c1]

The inventors synthesized Compound 4a, that is, 3-acetyl-8-(methylamino)-2H-benzo[g]chromene-2-one.

Specifically, a known synthesis starting material, that is, Compound d (20 mg, 0.1 mmol) and ethyl acetoacetate (25 μL, 0.2 mmol) were dissolved in ethanol (3 mL), and then piperidine (1 drop) was added thereto. The reaction mixture was stirred at 25° C. for 2 hours. After the reaction mixture was dried under reduced pressure, a crude product was purified by column chromatography in which it was passed through silica gel (eluent: EtOAc/CH₂Cl₂=5/95), thereby obtaining a red solid, Compound 4a (24 mg, 90%).

¹H NMR (CDCl₃, 300 MHz, 296 K): δ 8.58 (s, 1H), 7.94 (s, 1H), 7.67 (d, J=9.0 Hz, 1H), 7.41 (s, 1H), 6.84 (dd, J=9.0, 2.4 Hz, 1H), 6.69 (d, J=2.1 Hz, 1H), 4.38 (br, 1H), 2.99 (d, J=4.8 Hz, 3H), 2.73 (s, 3H).

### Example 21. Synthesis of Compound 4b; 3-acetyl-8-(pyrrolidine-1-yl)-2H-benzo[g]chromene-2-one

A general synthesis pathway of Compound 4b is shown in Reaction Scheme 21 below.

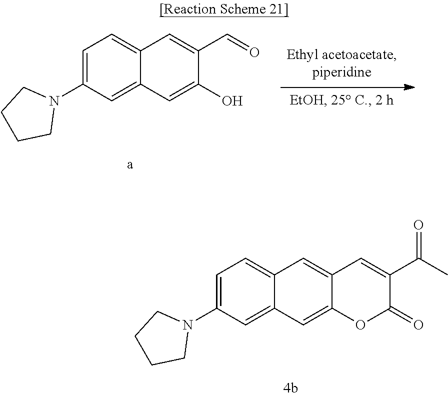 [SMILES: O=Cc1cc2ccc(N3CCCC3)cc2cc1O ; "EtOH, 25° C., 2 h" ; "Ethyl acetoacetate,piperidine" ; CC(=O)c1cc2cc3ccc(N4CCCC4)cc3cc2oc1=O]

The inventors synthesized Compound 4b, that is, 3-acetyl-8-(pyrrolidine-1-yl)-2H-benzo[g]chromene-2-one.

Specifically, a known synthesis starting material, that is, Compound a (24 mg, 0.1 mmol) and ethyl acetoacetate (25 μL, 0.2 mmol) were dissolved in ethanol (3 mL), and then piperidine (1 drop) was added thereto. The reaction mixture was stirred at 25° C. for 2 hours. After the reaction mixture was dried under reduced pressure, a crude product was purified by column chromatography in which it was passed through silica gel (eluent: EtOAc/CH₂Cl₂=5/95), thereby obtaining a red solid, Compound 4b (28 mg, 92%).

¹H NMR (CDCl₃, 300 MHz, 296 K): δ 8.54 (s, 1H), 7.91 (s, 1H), 7.71 (d, J=9.0 Hz, 1H), 7.34 (s, 1H), 6.96 (dd, J=9.0, 2.1 Hz, 1H), 6.63 (s, 1H), 3.45 (t, J=6.3 Hz, 4H), 2.72 (s, 3H), 2.07 (t, J=6.6 Hz, 4H).

### Example 22. Synthesis of Compound 4c; 1-(8-(methylamino)-2-oxo-2H-benzo[g]chromene-3-yl)butane-1,3-dione

A general synthesis pathway of Compound 4c is shown in Reaction Scheme 22 below.

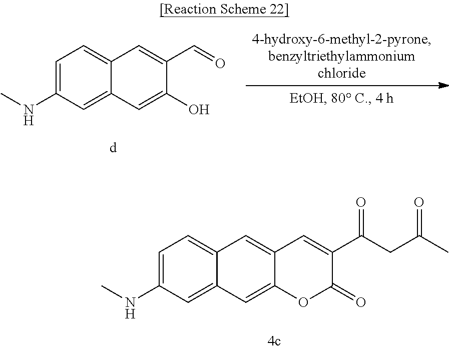 [SMILES: CNc1ccc2cc(C=O)c(O)cc2c1 ; "4-hydroxy-6-methyl-2-pyrone,benzyltriethylammoniumchloride" ; "EtOH, 80° C., 4 h" ; CNc1ccc2cc3cc(C(=O)CC(C)=O)c(=O)oc3cc2c1]

The inventors synthesized Compound 4c, that is, 1-(8-(methylamino)-2-oxo-2H-benzo[g]chromene-3-yl)butane-1,3-dione.

Specifically, a known synthesis starting material, that is, Compound d (200 mg, 1.0 mmol), 4-hydroxy-6-methyl-2-pyrone (230 mg, 1.2 mmol) and benzyltriethylammonium chloride (50 mg, 0.2 mmol) were dissolved in ethanol (10 mL). The reaction mixture was stirred at 80° C. for 4 hours. After the reaction mixture was dried under reduced pressure, a crude product was purified by column chromatography in which it was passed through silica gel (eluent: EtOAc/CH₂Cl₂=2/98), thereby obtaining a red solid, Compound 4c (284 mg, 92%).

¹H NMR (CDCl₃, 300 MHz, 296 K): δ 8.70 (s, 1H), 7.94 (s, 1H), 7.68 (d, J=9.0 Hz, 1H), 7.42 (s, 1H), 7.03 (s, 1H), 6.84 (dd, J=9.0, 2.4 Hz, 1H), 6.71 (d, J=2.1 Hz, 1H), 4.35 (br, 1H), 2.99 (d, J=5.1 Hz, 3H), 2.26 (s, 3H).

### Example 23. Synthesis of Compound 4d; 14842-(hydroxymethyl)pyrrolidine-1-yl)-2-oxo-2H-benzo[g]chromene-3-yl)butane-1,3-dione

A general synthesis pathway of Compound 4d is shown in Reaction Scheme 23 below.

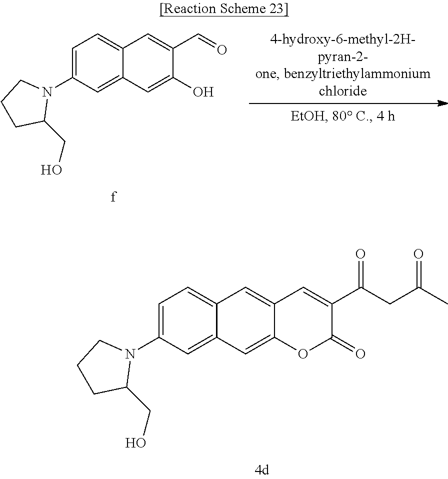 [SMILES: O=Cc1cc2ccc(N3CCCC3CO)cc2cc1O ; "4-hydroxy-6-methyl-2H-pyran-2-one, benzyltriethylammoniumchloride" ; "EtOH, 80° C., 4 h" ; CC(=O)CC(=O)c1cc2cc3ccc(N4CCCC4CO)cc3cc2oc1=O]

The inventors synthesized Compound 4d, that is, 1-(8-(2-(hydroxymethyl)pyrrolidine-1-yl)-2-oxo-2H-benzo[g]chromene-3-yl)butane-1,3-dione.

Specifically, a known synthesis starting material, that is, Compound f (100 mg, 0.37 mmol), 4-hydroxy-6-methyl-2-pyrone (230 mg, 1.2 mmol) and benzyltriethylammonium chloride (50 mg, 0.2 mmol) were dissolved in ethanol (10 mL). The reaction mixture was stirred at 80° C. for 4 hours. After the reaction mixture was dried under reduced pressure, a crude product was purified by column chromatography in which it was passed through silica gel (eluent: EtOAc/CH₂Cl₂=2/98), thereby obtaining a red solid, Compound 4d (105 mg, 75%).

¹H NMR (CDCl₃, 300 MHz, 296 K): δ 8.62 (s, 1H), 7.88 (s, 1H), 7.72 (d, J=9.0 Hz, 1H), 7.33 (s, 1H), 7.09 (d, J=8.7 Hz, 1H), 7.00 (s, 1H), 6.76 (d, J=2.1 Hz, 1H), 4.11 (br, 1H), 3.77-3.60 (m, 3H), 3.37-3.34 (m, 1H), 2.25 (s, 1H), 2.13 (br, 4H).

### Example 24. Synthesis of Compound 4e; 8-(allylamino)-3-benzoyl-2H-benzo[g]chromene-2-one

A general synthesis pathway of Compound 4e is shown in Reaction Scheme 24 below.

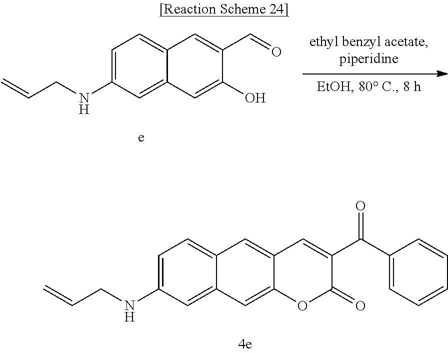 [SMILES: C=CCNc1ccc2cc(C=O)c(O)cc2c1 ; "EtOH, 80° C., 8 h" ; "ethyl benzyl acetate,piperidine" ; C=CCNc1ccc2cc3cc(C(=O)c4ccccc4)c(=O)oc3cc2c1]

The inventors synthesized Compound 4e, that is, 8-(allylamino)-3-benzoyl-2H-benzo[g]chromene-2-one.

Specifically, a known synthesis starting material, that is, Compound e (50 mg, 0.21 mmol) and ethyl benzyl acetate (43 μL, 0.25 mmol) were dissolved in ethanol (2 mL), and then piperidine (1 drop) was added thereto. The reaction mixture was stirred at 80° C. for 8 hours. The reaction mixture was dried under reduced pressure, thereby obtaining an orange solid, Compound 4e (72.4 mg, 97%).

¹H NMR (CDCl₃, 300 MHz, 296 K): δ 7.88 (s, 1H), 7.66 (m, 3H, J=23 Hz), 7.59 (m, 2H, J=11 Hz), 7.16 (m, 3H, J=22 Hz), 6.91 (d, 1H, J=9 Hz), 6.74 (s, 1H), 6.00 (m, 1H, J=38 Hz), 7.35 (d, 1H, J=18 Hz), 5.25 (d, 1H, J=11 Hz), 4.44 (t, 1H, J=11 Hz), 3.94 (d, 2H, J=8.1 Hz).

### Example 25. Synthesis of Compound 4f; 8-(allylamino)-3-(4-nitrobenzoyl)-2H-benzo[g]chromene-2-one

A general synthesis pathway of Compound 4f is shown in Reaction Scheme 25 below.

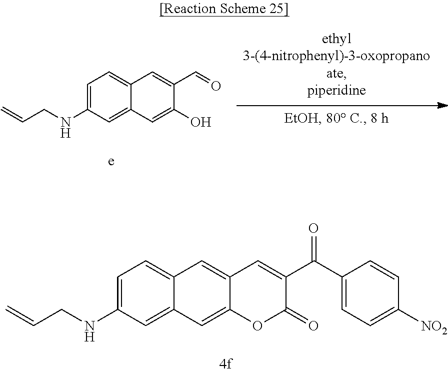 [SMILES: "ethyl3-(4-nitrophenyl)-3-oxopropanoate,piperidine" ; C=CCNc1ccc2cc(C=O)c(O)cc2c1 ; "EtOH, 80° C., 8 h" ; C=CCNc1ccc2cc3cc(C(=O)c4ccc([N+](=O)[O-])cc4)c(=O)oc3cc2c1]

The inventors synthesized Compound 4f, that is, 8-(allylamino)-3-(4-nitrobenzoyl)-2H-benzo[g]chromene-2-one.

Specifically, a known synthesis starting material, that is, Compound e (20 mg, 0.083 mmol) and ethyl 3-(4-nitrophenyl)-3-oxopropanoate (25 mg, 0.1 mmol) were dissolved in ethanol (2 mL), and then piperidine (1 drop) was added thereto. The reaction mixture was stirred at 80° C. for 8 hours. The reaction mixture was dried under reduced pressure, thereby obtaining an orange solid, Compound 4f (32.2 mg, 97%).

¹H NMR (CDCl₃, 300 MHz, 296 K): δ 8.58 (s, 1H), 8.32 (d, 2H, J=6.9 Hz), 8.24 (s, 1H), 8.09 (d, 2H, J=6.9 Hz), 7.77 (d, 1H, J=9 Hz), 7.47 (s, 1H), 7.05 (m, 2H, J=22 Hz), 6.76 (s, 1H), 5.96 (m, 1H, J=32 Hz), 5.30 (d, 1H, J=17 Hz), 5.17 (d, 1H, J=11 Hz), 3.86 (d, 2H, J=4.8 Hz).

### Example 26. Synthesis of Compound 42; 8-(allylamino)-3-(4-aminobenzoyl)-2H-benzo[g]chromene-2-one

A general synthesis pathway of Compound 4g is shown in Reaction Scheme 26 below.

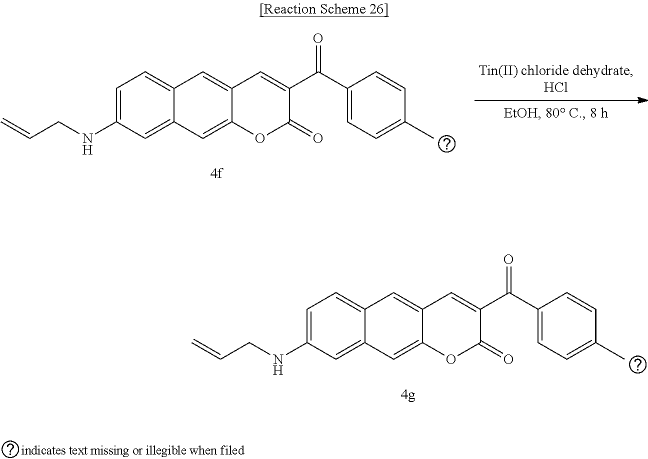

The inventors synthesized Compound 4g, that is, 8-(allylamino)-3-(4-aminobenzoyl)-2H-benzo[g]chromene-2-one.

Specifically, Compound 4f (20 mg, 0.054 mmol) synthesized in Example 25 was dissolved in ethanol (2 mL), and then tin(II) chloride dihydrate (77.6 mg, 0.34 mmol) and hydrochloric acid (0.12 mL) were added thereto. The mixture was stirred at 80° C. for 6 hours. After the reaction mixture was neutralized by adding 10% sodium carbonate dropwise, the reaction mixture was dried under reduced pressure, thereby obtaining an orange solid, Compound 4g (19.4 mg, 97%).

¹H NMR (CDCl₃, 300 MHz, 296 K): δ 8.04 (s, 1H), 7.86 (s, 1H), 7.78 (d, 2H, J=8.4 Hz), 7.68 (d, 1H, J=8.7 Hz), 7.45 (s, 1H), 6.90 (d, 1H, J=11 Hz), 6.77 (s, 1H), 6.66 (d, 2H, J=8.7 Hz), 6.00 (m, 1H, J=22 Hz), 5.36 (d, 1H, J=17 Hz), 5.26 (d, 1H, J=11 Hz), 3.94 (d, 2H, J=4.8 Hz).

### Example 27. Wavelengths of Maximum Absorbance of Benzocoumarin-Based Fluorescent Dyes

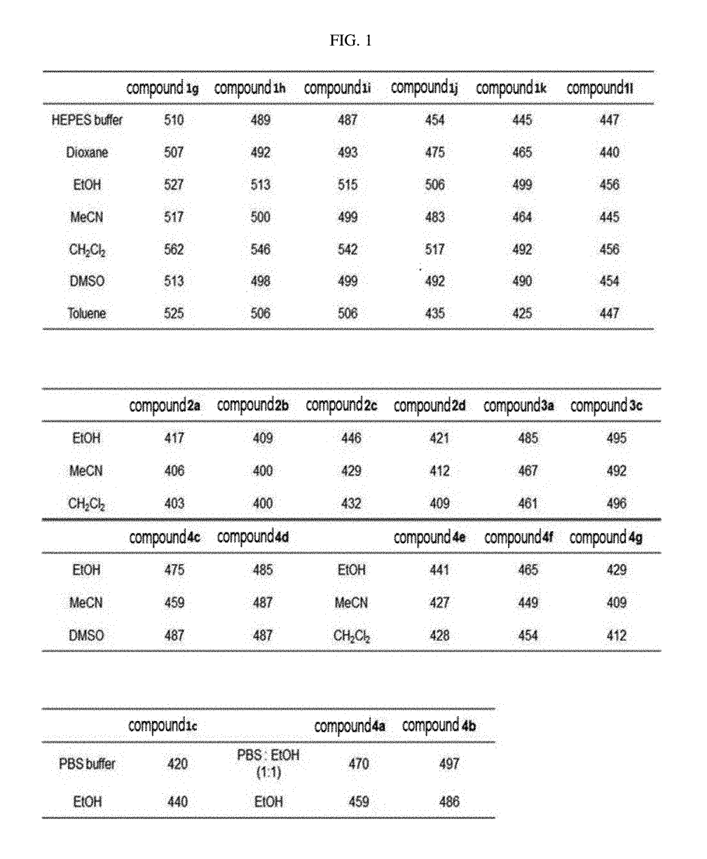

The inventors confirmed absorption characteristics of representative compounds among Compounds 1g to 1k, which are two-photon absorbing near-infrared fluorescent dyes, and Compounds 1a to 1f and 1l to 4g, which are two-photon absorbing fluorescent dyes, of the present invention, and the results are shown in FIG. 1.

Specifically, to confirm the characteristics of UV/Vis absorbance spectra of benzocoumarin-based near-infrared fluorescent dyes and derivatives thereof, the inventors measured UV/Vis absorbance spectra (measured using a HP8453 UV/Vis absorbance spectrophotometer) by charging a quartz cell with a 1-cm path length with 4-(2-hydroxyethyl)-1-piperazineethanesulfonic acid (HEPES) buffer, phosphate-buffered saline (PBS) buffer, dioxane, ethanol, acetonitrile, dichloromethane, dimethylsulfoxide, or a toluene solution (containing 1% DMSO), which contains 10 μM each of Compounds 1a to 4g, and the wavelength of maximum absorbance is shown in FIG. 1. Meanwhile, the wavelengths of maximum absorbance of Compounds 1a, 1b, 1d, 1e and 1f did not have a significant difference from those of Compounds 1c and 1g to 1l.

### Example 28. Wavelengths of Maximum Absorbance of Benzocoumarin-Based Fluorescent Dyes

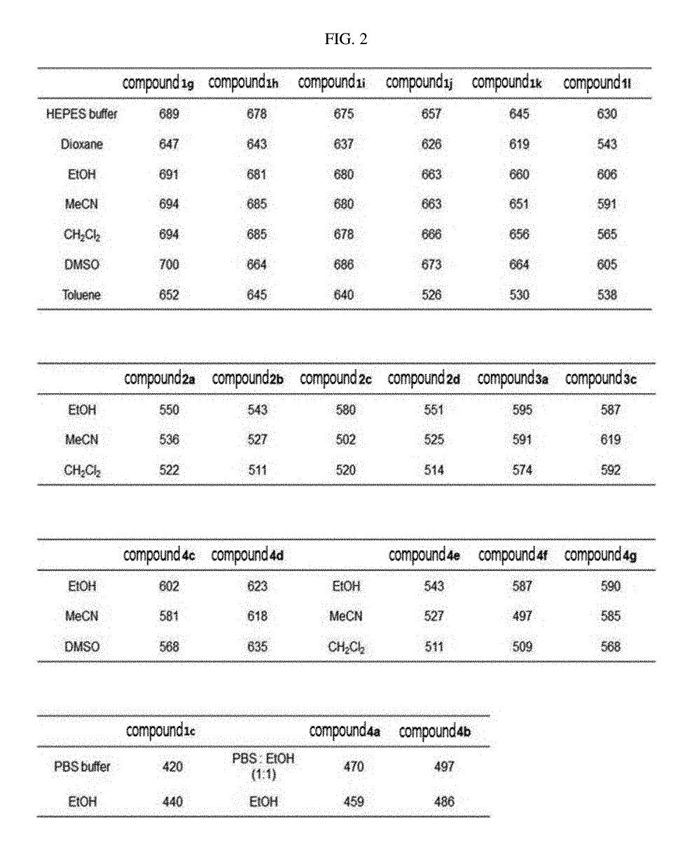

The inventors confirmed absorption characteristics of representative compounds among Compounds 1g to 1k, which are two-photon absorbing near-infrared fluorescent dyes, and Compounds 1a to 1f and 1l to 4g, which are two-photon absorbing fluorescent dyes, of the present invention, and the results are shown in FIG. 2.

Specifically, to confirm the characteristics of fluorescent spectra of two-photon absorbing near-infrared fluorescent dyes, the inventors measured fluorescence spectra (measured using a photon technical international fluorescence system) by charging a quartz cell with a 1-cm path length with 4-(2-hydroxyethyl)-1-piperazineethanesulfonic acid (HEPES) buffer, (2-hydroxyethyl)-1-piperazineethanesulfonic acid buffer, dioxane, ethanol, acetonitrile, dichloromethane, dimethylsulfoxide, a toluene solution (containing 1% DMSO), which contains 10 μM each of Compounds 1a to 4g, and the wavelength of maximum absorbance is shown in FIG. 2. Meanwhile, the wavelengths of maximum absorbance of Compounds 1a, 1b, 1d, 1e and 1f did not have a significant difference from those of Compounds 1c and 1g to 1l.

### Example 29. Observation of Two-Photon Fluorescent Microscope Image of Mouse Tissue Treated with Compound 1g

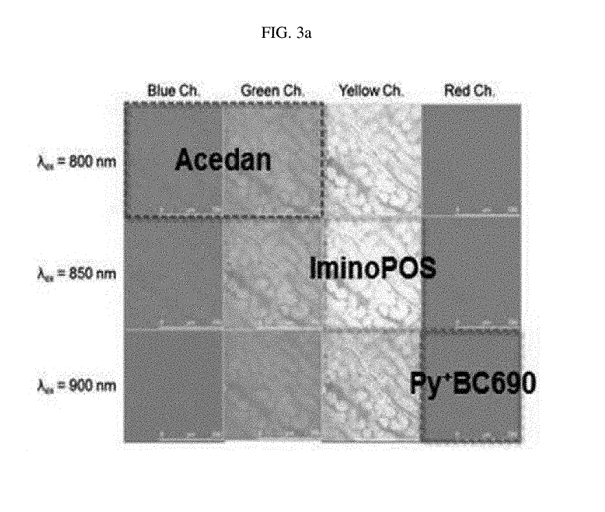

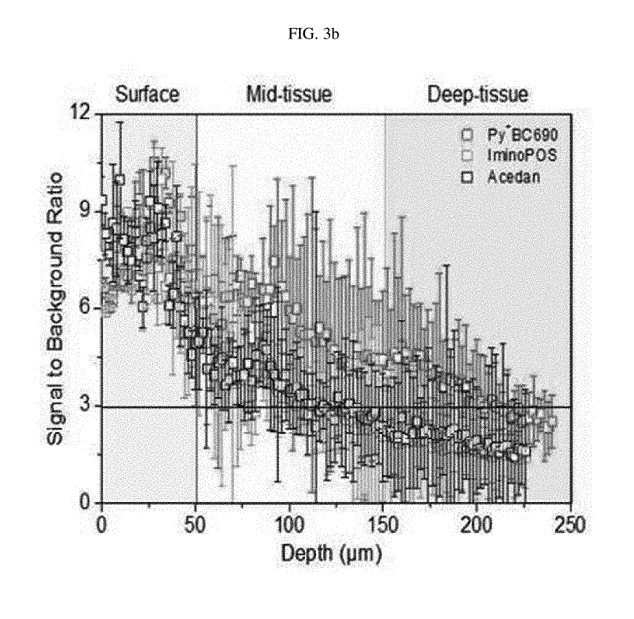

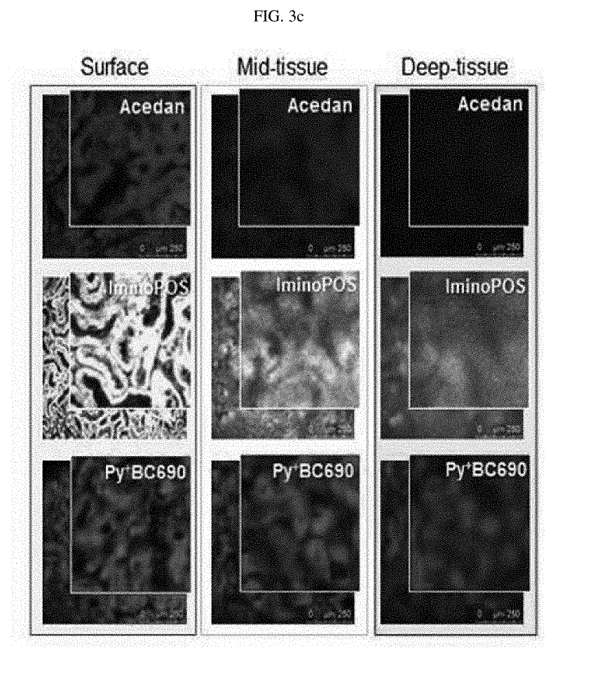

The inventors observed fluorescent changes by two-photon microscopy after mouse tissue was treated with Compound 1g of the present invention, and the result is shown in FIG. 3.

Specifically, to observe a two-photon microscopy image of mouse tissue treated with Compound 1g, kidney tissue of a Balb/C type mouse (6 weeks old) was used, and an experiment was carried out under a light-protected condition (dark room). The mouse kidney tissue was dissected and washed with a PBS buffer solution, and then each organ was frozen over dry-ice for 5 minutes. The frozen organs were crushed with a hammer, and then a tissue slice sample was prepared to a thickness of 16 μm using a cutter (Cryostat machine, Leica, CM3000 model). To fix the organ to the cutter, an optical cutting temperature (OCT) compound, 10% polyvinyl alcohol, 25% polyethylene glycol, 85.5% inactive species were used. The tissue slice sample was put on a specimen block (Paul Marienfeld GMbH & Co.), and the specimen block was immersed in 4% paraformaldehyde for 10 minutes and washed with a PBS buffer solution, and then the tissue was fixed again using a mount solution (Gel Mount, BIOMEDA). The prepared tissue slice sample was immersed in a PBS buffer solution containing Compound 1g and acedan or IminoPOS at a concentration of 10 μM for 10 minutes, washed with a PBS buffer solution three times, and then fixed with 4% paraformaldehyde, followed by observation of fluorescence. A two-photon electron microscope consisted of an upright microscope (BX51, Olympus) and 20× and 40× objective lenses (XLUMPLEN, NA 1.0, Olympus), and titanium: sapphire laser (Chameleon Ultra II, Coherent) was used.

As a result, as shown in FIG. 3a, two-photon excitation wavelengths at 800 nm (acedan), 850 nm (IminoPOS), and 900 nm (Compound 1g) were observed at gradually increasing laser output power from 5 mW to 95 mW.

In addition, as shown in FIG. 3b, it can be confirmed that a signal/noise (S/N) ratio was increased in the image obtained using Compound 1g obtained by tissue imaging from a surface layer to a middle depth (50 to 150 μm), compared to the images obtained using acedan and IminoPOS.

Moreover, as shown in FIG. 3c, it can be confirmed that a high-definition and high-contrast image was obtained when Compound 1g was used, compared to when acedan or IminoPOS was used.

A two-photon absorbing benzocoumarin-based fluorescent dye of the present invention can absorb or emit light in a longer wavelength range than conventional two-photon absorbing fluorescent dyes such as a 7-aminocoumarin derivative, acedan, naphthalimide, etc., and the benzocoumarin-based near-infrared fluorescent dye can minimize interference of autofluorescence and obtain a clear image with high resolution, and therefore it is expected to be effectively used in imaging studies.

The two-photon absorbing near-infrared fluorescent dye of the present invention is also expected to be suitable for high-resolution imaging of deep tissue.

It would be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative and not limited in any aspect.

What is claimed is:

1. A pyridinium-benzocoumarin (Py⁺BC) derivative compound represented by Formula 1 and its precursor, a pyridinyl-benzocoumarin (PyBC) derivative compound, an aryl-benzocoumarin (ArBC) derivative compound represented by Formula 2, a benzothiazolyl-benzocoumarin (BtBC) derivative compound represented by Formula 3, a keto-benzocoumarin (ketoBC) derivative compound represented by Formula 4, or a pharmaceutically acceptable salt thereof: 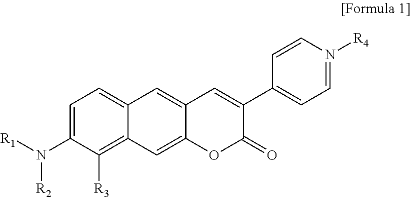
where R₁ and R₂ are each independently hydrogen (H), a methyl group (Me), an allyl group, or a C₂-C₁₂ unsubstituted alkyl group;
R₁ is 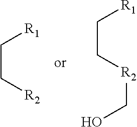
linked to R₂ in a ring;
R₃ is hydrogen (H), or 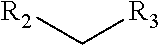
linked to R₂ in a ring; and
R₄ is hydrogen (H), a methyl (Me) group, or oxygen (O). 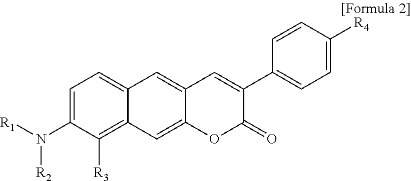
where R₁ and R₂ are each independently hydrogen (H), a methyl group (Me), an allyl group, or a C₂-C₁₂ unsubstituted alkyl group;
R₁ is 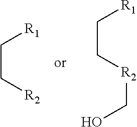
linked to R₂ in a ring;
R₃ is hydrogen (H), or 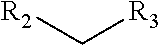
linked to R₂ in a ring; and
R₄ is a hydroxyl group (OH), an amine group (NH₂), a nitro group (NO₂), an acetate group (OCOMe), or a carboxyl group (COOH). 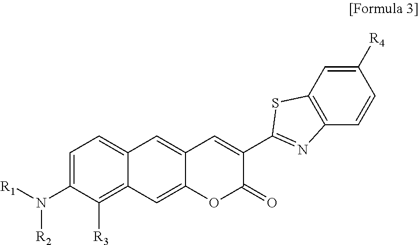
where R₁ and R₂ are each independently hydrogen (H), a methyl group (Me), an allyl group, or a C₂-C₁₂ unsubstituted alkyl group;
R₁ is 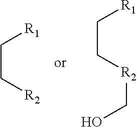
linked to R₂ in a ring;
R₃ is hydrogen (H), or 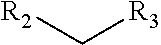
linked to R₂ in a ring; and
R₄ is a hydroxyl group (OH), an amine group (NH₂), a nitro group (NO₂), or a carboxyl group (COOH). 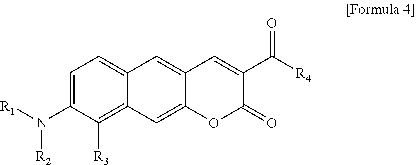
where R₁ and R₂ are each independently hydrogen (H), a methyl group (Me), an allyl group, or a C₂-C₁₂ unsubstituted alkyl group;
R₁ is 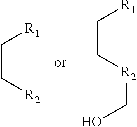
linked to R₂ in a ring;
R₃ is hydrogen (H), or 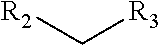
linked to R₂ in a ring;
R₄ is hydrogen (H), a methyl group (Me), an acetoxy group (COMe), or 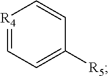
and
R₅ is hydrogen (H), a hydroxyl group (OH), an amine group (NH₂), or a nitro group (NO₂).

2. The compound or pharmaceutically acceptable salt thereof of claim 1, wherein the compound is a compound represented by one formula selected from the group consisting of Formulas 1g to 1k below.

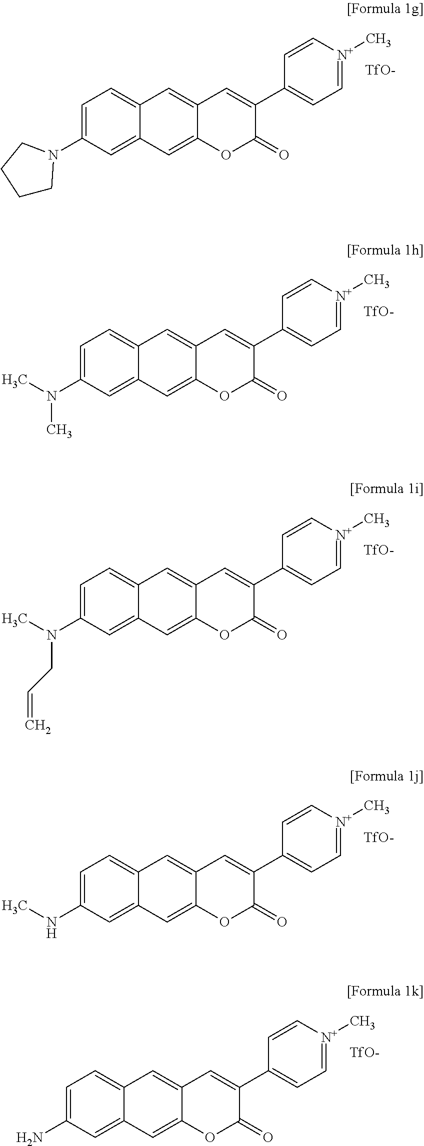

3. The compound or pharmaceutically acceptable salt thereof of claim 1, wherein the compound is a compound represented by Formula 1g.

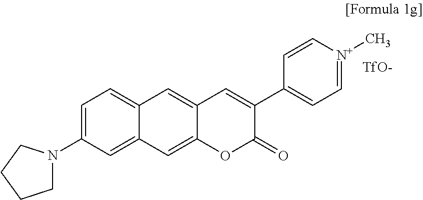

4. The compound or pharmaceutically acceptable salt thereof of claim 1, wherein the compound is a two-photon absorbing fluorescent dye.

5. The compound or pharmaceutically acceptable salt thereof of claim 2, wherein the compound is a two-photon absorbing near-infrared fluorescent dye.

6. A composition for cell or tissue imaging, comprising:
the compound or pharmaceutically acceptable salt thereof of claim 1.

7. A composition for cell or tissue imaging, comprising:
the compound or pharmaceutically acceptable salt thereof of claim 2.

8. A composition for cell or tissue imaging, comprising:
the compound or pharmaceutically acceptable salt thereof of claim 3.

9. A composition for cell or tissue imaging, comprising:
the compound or pharmaceutically acceptable salt thereof of claim 4.

10. A composition for cell or tissue imaging, comprising:
the compound or pharmaceutically acceptable salt thereof of claim 5.
